(19) United States
(12) Patent Application Publication
(10) Pub. No.: US 2021/0290168 A1
(43) Pub. Date: Sep. 23, 2021

(54) Compensation for Delays in an Optical Measurement System

(71) Applicant: HI LLC, Los Angeles, CA (US)

(72) Inventors: Bruno Do Valle, Brighton, MA (US); Rong Jin, Acton, MA (US); Jacob Dahle, Arlington, MA (US); Husam Katnani, Braintree, MA (US); Ryan Field, Culver City, CA (US)

(21) Appl. No.: 17/202,539

(22) Filed: Mar. 16, 2021

Related U.S. Application Data
(60) Provisional application No. 62/992,481, filed on Mar. 20, 2020.
(60) Provisional application No. 63/042,236, filed on Jun. 22, 2020.

(51) Int. Cl. A61B 5/00

(52) U.S. Cl. CPC A61B 5/7214; A61B 5/6803; A61B 2576/026; A61B 2562/0238; A61B 2562/046; A61B 5/0082

(57) ABSTRACT

An exemplary system includes a first photodetector configured to detect a first photon via a first path from a light source and a second photodetector configured to detect a second photon via a second path. The system includes a control system configured to record a first time based on the first photodetector detecting the first photon, the first time including a delay between the first photodetector detecting the first photon and the control system receiving a photodetector output pulse from the first photodetector. The control system is configured to record a second time based on the second photodetector detecting the second photon, the second time including a delay between the second photodetector and the control system. The control system is configured to determine, based on the first and second time, an amount of time for the second photon to travel from the light source to the second photodetector.

### RELATED APPLICATIONS

The present application claims priority under 35 U.S.C. § 119(e) to U.S. Provisional Patent Application No. 62/992,481, filed on Mar. 20, 2020, and to U.S. Provisional Patent Application No. 63/042,236, filed on Jun. 22, 2020. These applications are incorporated herein by reference in their respective entireties.

### BACKGROUND INFORMATION

Detecting neural activity in the brain (or any other turbid medium) is useful for medical diagnostics, imaging, neuroengineering, brain-computer interfacing, and a variety of other diagnostic and consumer-related applications. For example, it may be desirable to detect neural activity in the brain of a user to determine if a particular region of the brain has been impacted by reduced blood irrigation, a hemorrhage, or any other type of damage. As another example, it may be desirable to detect neural activity in the brain of a user and computationally decode the detected neural activity into commands that can be used to control various types of consumer electronics (e.g., by controlling a cursor on a computer screen, changing channels on a television, turning lights on, etc.).

Neural activity and other attributes of the brain may be determined or inferred by measuring responses of tissue within the brain to light pulses. One technique to measure such responses is time-correlated single-photon counting (TCSPC). Time-correlated single-photon counting detects single photons and measures a time of arrival of the photons with respect to a reference signal (e.g., a light source). By repeating the light pulses, TCSPC may accumulate a sufficient number of photon events to statistically determine a histogram representing the distribution of detected photons. Based on the histogram of photon distribution, the response of tissue to light pulses may be determined in order to study the detected neural activity and/or other attributes of the brain.

A photodetector capable of detecting a single photon (i.e., a single particle of optical energy) is an example of a non-invasive detector that can be used in an optical measurement system to detect neural activity within the brain. An exemplary photodetector is implemented by a semiconductor-based single-photon avalanche diode (SPAD), which is capable of capturing individual photons with very high time-of-arrival resolution (a few tens of picoseconds). With such high time resolutions, effects of circuit propagation delays and other system delays may become significant.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an exemplary optical measurement system.

FIG. 2 illustrates an exemplary detector architecture.

FIG. 3 illustrates an exemplary timing diagram for performing an optical measurement operation using an optical measurement system.

FIG. 4 illustrates a graph of an exemplary temporal point spread function that may be generated by an optical measurement system in response to a light pulse.

FIG. 5 shows an exemplary non-invasive wearable brain interface system.

FIG. 6 shows an exemplary wearable module assembly.

FIG. 7 shows an exemplary signal path diagram.

FIG. 8 shows an exemplary timing diagram.

FIG. 9 illustrates an exemplary configuration of an optical measurement system.

FIG. 10 shows an exemplary timing diagram.

FIG. 11 illustrates an exemplary configuration of an optical measurement system.

FIGS. 12-13 illustrate exemplary histograms.

FIGS. 14-15 show exemplary configurations of optical measurement system components.

FIGS. 16-21 illustrate embodiments of a wearable device that includes elements of the optical measurement systems described herein.

FIG. 22 illustrates an exemplary computing device.

FIGS. 23-26 illustrate exemplary methods.

### DETAILED DESCRIPTION

Systems and methods for compensation for delays in an optical measurement system are described herein.

For example, the systems and methods described herein may be configured to determine various propagation delays introduced by components and/or circuits of the optical measurement system. Propagation delays may vary based on variable conditions as well as variations within components or between components. Such delays may skew photon detection times by the optical measurement system resulting in errors, especially in high resolution timing applications. The various systems and methods described herein to determine and/or compensate for various propagation delays may provide various benefits and advantages compared to conventional systems. For example, some of the systems and methods may allow the optical measurement system to determine propagation delays and compensate accordingly. Some of the systems and methods may allow the optical measurement system to continuously adjust for propagation delays that may vary depending on varying conditions.

These and other advantages and benefits of the present systems, circuits, and methods are described more fully herein.

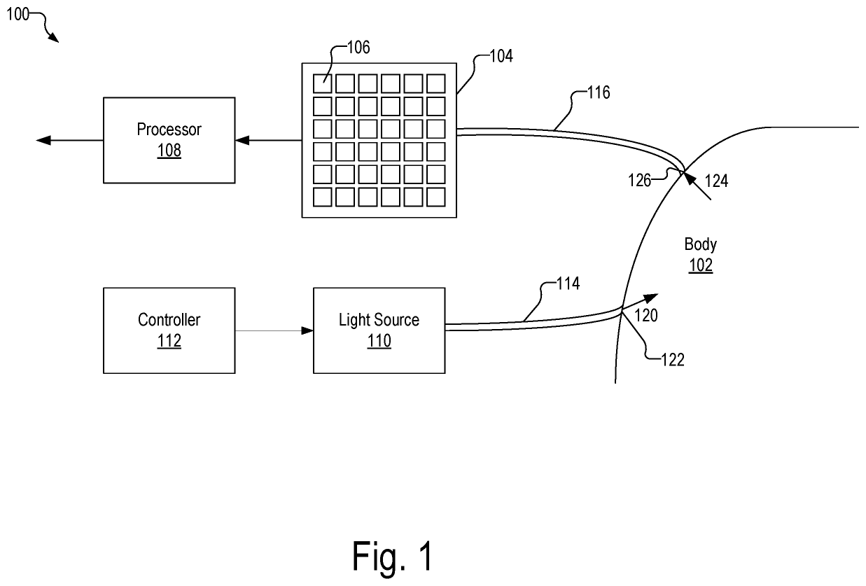

FIG. 1 shows an exemplary optical measurement system 100 configured to perform an optical measurement operation with respect to a body 102. Optical measurement system 100 may, in some examples, be portable and/or wearable by a user. Optical measurement systems that may be used in connection with the embodiments described herein are described more fully in U.S. patent application Ser. No. 17/176,315, filed Feb. 16, 2021; U.S. patent application Ser. No. 17/176,309, filed Feb. 16, 2021; U.S. patent application Ser. No. 17/176,460, filed Feb. 16, 2021; U.S. patent application Ser. No. 17/176,470, filed Feb. 16, 2021; U.S. patent application Ser. No. 17/176,487, filed Feb. 16, 2021; U.S. patent application Ser. No. 17/176,539, filed Feb. 16, 2021; U.S. patent application Ser. No. 17/176,560, filed Feb. 16, 2021; and U.S. patent application Ser. No. 17/176,466, filed Feb. 16, 2021, which applications are incorporated herein by reference in their entirety.

In some examples, optical measurement operations performed by optical measurement system 100 are associated with a time domain-based optical measurement technique. Example time domain-based optical measurement techniques include, but are not limited to, TCSPC, time domain near infrared spectroscopy (TD-NIRS), time domain diffusive correlation spectroscopy (TD-DCS), and time domain Digital Optical Tomography (TD-DOT).

As shown, optical measurement system 100 includes a detector 104 that includes a plurality of individual photodetectors (e.g., photodetector 106), a processor 108 coupled to detector 104, a light source 110, a controller 112, and optical conduits 114 and 116 (e.g., light pipes). However, one or more of these components may not, in certain embodiments, be considered to be a part of optical measurement system 100. For example, in implementations where optical measurement system 100 is wearable by a user, processor 108 and/or controller 112 may in some embodiments be separate from optical measurement system 100 and not configured to be worn by the user.

Detector 104 may include any number of photodetectors 106 as may serve a particular implementation, such as 2ⁿ photodetectors (e.g., 256, 512, . . . , 16384, etc.), where n is an integer greater than or equal to one (e.g., 4, 5, 8, 10, 11, 14, etc.). Photodetectors 106 may be arranged in any suitable manner.

Photodetectors 106 may each be implemented by any suitable circuit configured to detect individual photons of light incident upon photodetectors 106. For example, each photodetector 106 may be implemented by a single photon avalanche diode (SPAD) circuit and/or other circuitry as may serve a particular implementation.

Processor 108 may be implemented by one or more physical processing (e.g., computing) devices. In some examples, processor 108 may execute instructions (e.g., software) configured to perform one or more of the operations described herein.

Light source 110 may be implemented by any suitable component configured to generate and emit light. For example, light source 110 may be implemented by one or more laser diodes, distributed feedback (DFB) lasers, super luminescent diodes (SLDs), light emitting diodes (LEDs), diode-pumped solid-state (DPSS) lasers, super luminescent light emitting diodes (sLEDs), vertical-cavity surface-emitting lasers (VCSELs), titanium sapphire lasers, micro light emitting diodes (mLEDs), and/or any other suitable laser or light source. In some examples, the light emitted by light source 110 is high coherence light (e.g., light that has a coherence length of at least 5 centimeters) at a predetermined center wavelength.

Light source 110 is controlled by controller 112, which may be implemented by any suitable computing device (e.g., processor 108), integrated circuit, and/or combination of hardware and/or software as may serve a particular implementation. In some examples, controller 112 is configured to control light source 110 by turning light source 110 on and off and/or setting an intensity of light generated by light source 110. Controller 112 may be manually operated by a user, or may be programmed to control light source 110 automatically.

Light emitted by light source 110 may travel via an optical conduit 114 (e.g., a light pipe, a light guide, a waveguide, a single-mode optical fiber, and/or or a multi-mode optical fiber) to body 102 of a subject. In cases where optical conduit 114 is implemented by a light guide, the light guide may be spring loaded and/or have a cantilever mechanism to allow for conformably pressing the light guide firmly against body 102.

Body 102 may include any suitable turbid medium. For example, in some implementations, body 102 is a head or any other body part of a human or other animal. Alternatively, body 102 may be a non-living object. For illustrative purposes, it will be assumed in the examples provided herein that body 102 is a human head.

As indicated by arrow 120, the light emitted by light source 110 enters body 102 at a first location 122 on body 102. Accordingly, a distal end of optical conduit 114 may be positioned at (e.g., right above or physically attached to) first location 122 (e.g., to a scalp of the subject). In some examples, the light may emerge from optical conduit 114 and spread out to a certain spot size on body 102 to fall under a predetermined safety limit.

As used herein, “distal” means nearer, along the optical path of the light emitted by light source 110 or the light received by detector 104, to the target (e.g., within body 102) than to light source 110 or detector 104. Thus, the distal end of optical conduit 114 is nearer to body 102 than to light source 110, and the distal end of optical conduit 116 is nearer to body 102 than to detector 104. Additionally, as used herein, “proximal” means nearer, along the optical path of the light emitted by light source 110 or the light received by detector 104, to light source 110 or detector 104 than to body 102. Thus, the proximal end of optical conduit 114 is nearer to light source 110 than to body 102, and the proximal end of optical conduit 116 is nearer to detector 104 than to body 102.

As shown, the distal end of optical conduit 116 (e.g., a light pipe, a single-mode optical fiber, and/or or a multi-mode optical fiber) is positioned at (e.g., right above or physically attached to) output location 126 on body 102. In this manner, optical conduit 116 may collect light 124 as it exits body 102 at location 126 and carry the light to detector 104. The light may pass through one or more lenses and/or other optical elements (not shown) that direct the light onto each of the photodetectors 106 included in detector 104.

Photodetectors 106 may be connected in parallel in detector 104. An output of each of photodetectors 106 may be accumulated to generate an accumulated output of detector 104. Processor 108 may receive the accumulated output and determine, based on the accumulated output, a temporal distribution of photons detected by photodetectors 106. Processor 108 may then generate, based on the temporal distribution, a histogram representing a light pulse response of a target (e.g., brain tissue, blood flow, etc.) in body 102. Example embodiments of accumulated outputs are described herein.

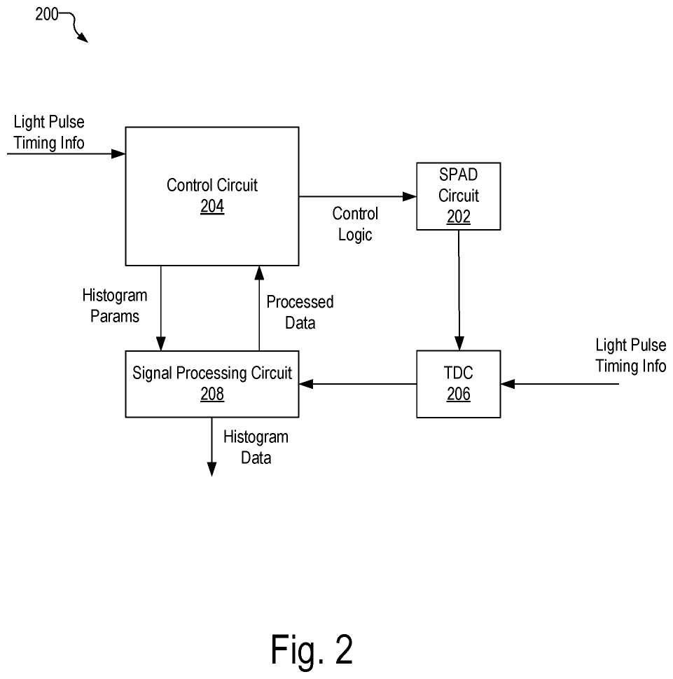

FIG. 2 illustrates an exemplary detector architecture 200 that may be used in accordance with the systems and methods described herein. As shown, architecture 200 includes a SPAD circuit 202 that implements photodetector 106, a control circuit 204, a time-to-digital converter (TDC) 206, and a signal processing circuit 208. Architecture 200 may include additional or alternative components as may serve a particular implementation.

In some examples, SPAD circuit 202 includes a SPAD and a fast gating circuit configured to operate together to detect a photon incident upon the SPAD. As described herein, SPAD circuit 202 may generate an output when SPAD circuit 202 detects a photon.

The fast gating circuit included in SPAD circuit 202 may be implemented in any suitable manner. For example, the fast gating circuit may include a capacitor that is pre-charged with a bias voltage before a command is provided to arm the SPAD. Gating the SPAD with a capacitor instead of with an active voltage source, such as is done in some conventional SPAD architectures, has a number of advantages and benefits. For example, a SPAD that is gated with a capacitor may be armed practically instantaneously compared to a SPAD that is gated with an active voltage source. This is because the capacitor is already charged with the bias voltage when a command is provided to arm the SPAD. This is described more fully in U.S. Pat. Nos. 10,158,038 and 10,424,683, which are incorporated herein by reference in their entireties.

In some alternative configurations, SPAD circuit 202 does not include a fast gating circuit. In these configurations, the SPAD included in SPAD circuit 202 may be gated in any suitable manner or be configured to operate in a free running mode with passive quenching.

Control circuit 204 may be implemented by an application specific integrated circuit (ASIC) or any other suitable circuit configured to control an operation of various components within SPAD circuit 202. For example, control circuit 204 may output control logic that puts the SPAD included in SPAD circuit 202 in either an armed or a disarmed state.

In some examples, control circuit 204 may control a gate delay, which specifies a predetermined amount of time control circuit 204 is to wait after an occurrence of a light pulse (e.g., a laser pulse) to put the SPAD in the armed state. To this end, control circuit 204 may receive light pulse timing information, which indicates a time at which a light pulse occurs (e.g., a time at which the light pulse is applied to body 102). Control circuit 204 may also control a programmable gate width, which specifies how long the SPAD is kept in the armed state before being disarmed.

Control circuit 204 is further configured to control signal processing circuit 208. For example, control circuit 204 may provide histogram parameters (e.g., time bins, number of light pulses, type of histogram, etc.) to signal processing circuit 208. Signal processing circuit 208 may generate histogram data in accordance with the histogram parameters. In some examples, control circuit 204 is at least partially implemented by controller 112.

TDC 206 is configured to measure a time difference between an occurrence of an output pulse generated by SPAD circuit 202 and an occurrence of a light pulse. To this end, TDC 206 may also receive the same light pulse timing information that control circuit 204 receives. TDC 206 may be implemented by any suitable circuitry as may serve a particular implementation.

Signal processing circuit 208 is configured to perform one or more signal processing operations on data output by TDC 206. For example, signal processing circuit 208 may generate histogram data based on the data output by TDC 206 and in accordance with histogram parameters provided by control circuit 204. To illustrate, signal processing circuit 208 may generate, store, transmit, compress, analyze, decode, and/or otherwise process histograms based on the data output by TDC 206. In some examples, signal processing circuit 208 may provide processed data to control circuit 204, which may use the processed data in any suitable manner. In some examples, signal processing circuit 208 is at least partially implemented by processor 108.

In some examples, each photodetector 106 (e.g., SPAD circuit 202) may have a dedicated TDC 206 associated therewith. For example, for an array of N photodetectors 106, there may be a corresponding array of N TDCs 206. Alternatively, a single TDC 206 may be associated with multiple photodetectors 106. Likewise, a single control circuit 204 and a single signal processing circuit 208 may be provided for one or more photodetectors 106 and/or TDCs 206.

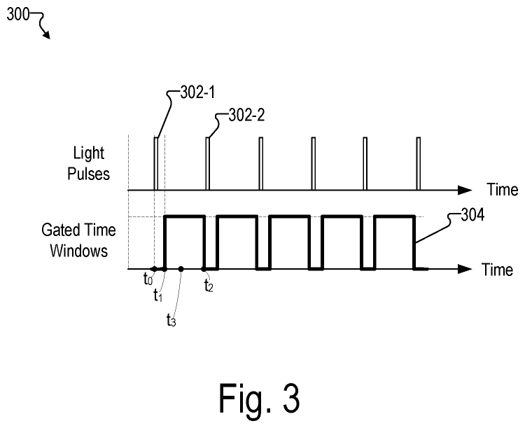

FIG. 3 illustrates an exemplary timing diagram 300 for performing an optical measurement operation using optical measurement system 100. Optical measurement system 100 may be configured to perform the optical measurement operation by directing light pulses (e.g., laser pulses) toward a target within a body (e.g., body 102). The light pulses may be short (e.g., 10-2000 picoseconds (ps)) and repeated at a high frequency (e.g., between 100,000 hertz (Hz) and 100 megahertz (MHz)). The light pulses may be scattered by the target and then detected by optical measurement system 100. Optical measurement system 100 may measure a time relative to the light pulse for each detected photon. By counting the number of photons detected at each time relative to each light pulse repeated over a plurality of light pulses, optical measurement system 100 may generate a histogram that represents a light pulse response of the target (e.g., a temporal point spread function (TPSF)). The terms histogram and TPSF are used interchangeably herein to refer to a light pulse response of a target.

For example, timing diagram 300 shows a sequence of light pulses 302 (e.g., light pulses 302-1 and 302-2) that may be applied to the target (e.g., tissue within a brain of a user, blood flow, a fluorescent material used as a probe in a body of a user, etc.). Timing diagram 300 also shows a pulse wave 304 representing predetermined gated time windows (also referred as gated time periods) during which photodetectors 106 are gated ON to detect photons. Referring to light pulse 302-1, light pulse 302-1 is applied at a time t₀. At a time t₁, a first instance of the predetermined gated time window begins. Photodetectors 106 may be armed at time t₁, enabling photodetectors 106 to detect photons scattered by the target during the predetermined gated time window. In this example, time t₁ is set to be at a certain time after time t₀, which may minimize photons detected directly from the laser pulse, before the laser pulse reaches the target. However, in some alternative examples, time t₁ is set to be equal to time t₀.

At a time t₂, the predetermined gated time window ends. In some examples, photodetectors 106 may be disarmed at time t₂. In other examples, photodetectors 106 may be reset (e.g., disarmed and re-armed) at time t₂ or at a time subsequent to time t₂. During the predetermined gated time window, photodetectors 106 may detect photons scattered by the target. Photodetectors 106 may be configured to remain armed during the predetermined gated time window such that photodetectors 106 maintain an output upon detecting a photon during the predetermined gated time window. For example, a photodetector 106 may detect a photon at a time t₃, which is during the predetermined gated time window between times t₁ and t₂. The photodetector 106 may be configured to provide an output indicating that the photodetector 106 has detected a photon. The photodetector 106 may be configured to continue providing the output until time t₂, when the photodetector may be disarmed and/or reset. Optical measurement system 100 may generate an accumulated output from the plurality of photodetectors. Optical measurement system 100 may sample the accumulated output to determine times at which photons are detected by photodetectors 106 to generate a TPSF.

As mentioned, in some alternative examples, photodetector 106 may be configured to operate in a free-running mode such that photodetector 106 is not actively armed and disarmed (e.g., at the end of each predetermined gated time window represented by pulse wave 304). In contrast, while operating in the free-running mode, photodetector 106 may be configured to reset within a configurable time period after an occurrence of a photon detection event (i.e., after photodetector 106 detects a photon) and immediately begin detecting new photons. However, only photons detected within a desired time window (e.g., during each gated time window represented by pulse wave 304) may be included in the TPSF.

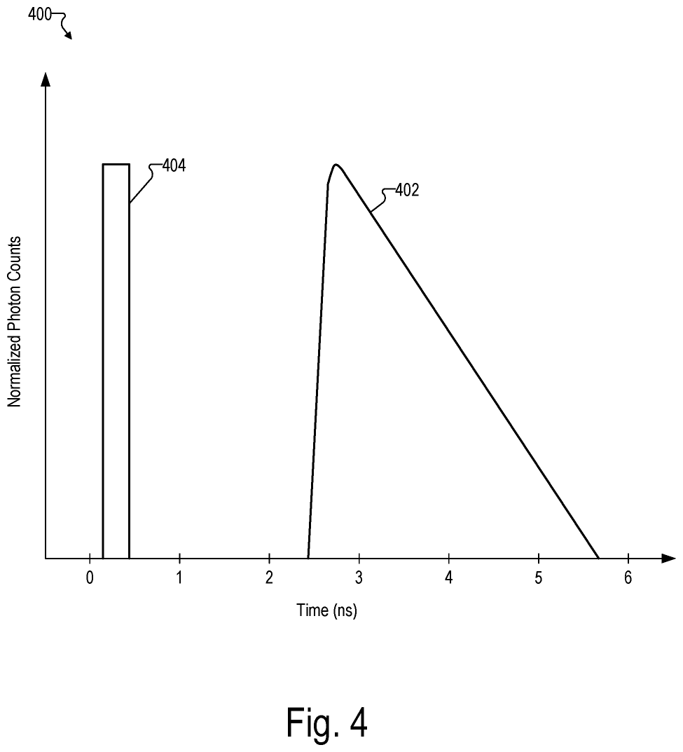

FIG. 4 illustrates a graph 400 of an exemplary TPSF 402 that may be generated by optical measurement system 100 in response to a light pulse 404 (which, in practice, represents a plurality of light pulses). Graph 400 shows a normalized count of photons on a y-axis and time bins on an x-axis. As shown, TPSF 402 is delayed with respect to a temporal occurrence of light pulse 404. In some examples, the number of photons detected in each time bin subsequent to each occurrence of light pulse 404 may be aggregated (e.g., integrated) to generate TPSF 402. TPSF 402 may be analyzed and/or processed in any suitable manner to determine or infer detected neural activity.

Optical measurement system 100 may be implemented by or included in any suitable device. For example, optical measurement system 100 may be included, in whole or in part, in a non-invasive wearable device (e.g., a headpiece) that a user may wear to perform one or more diagnostic, imaging, analytical, and/or consumer-related operations. The non-invasive wearable device may be placed on a user's head or other part of the user to detect neural activity. In some examples, such neural activity may be used to make behavioral and mental state analysis, awareness and predictions for the user.

Mental state described herein refers to the measured neural activity related to physiological brain states and/or mental brain states, e.g., joy, excitement, relaxation, surprise, fear, stress, anxiety, sadness, anger, disgust, contempt, contentment, calmness, focus, attention, approval, creativity, positive or negative reflections/attitude on experiences or the use of objects, etc. Further details on the methods and systems related to a predicted brain state, behavior, preferences, or attitude of the user, and the creation, training, and use of neuromes can be found in U.S. Provisional Patent Application No. 63/047,991, filed Jul. 3, 2020. Exemplary measurement systems and methods using biofeedback for awareness and modulation of mental state are described in more detail in U.S. patent application Ser. No. 16/364,338, filed Mar. 26, 2019, published as US2020/0196932A1. Exemplary measurement systems and methods used for detecting and modulating the mental state of a user using entertainment selections, e.g., music, film/video, are described in more detail in U.S. patent application Ser. No. 16/835,972, filed Mar. 31, 2020, published as US2020/0315510A1. Exemplary measurement systems and methods used for detecting and modulating the mental state of a user using product formulation from, e.g., beverages, food, selective food/drink ingredients, fragrances, and assessment based on product-elicited brain state measurements are described in more detail in U.S. patent application Ser. No. 16/853,614, filed Apr. 20, 2020, published as US2020/0337624A1. Exemplary measurement systems and methods used for detecting and modulating the mental state of a user through awareness of priming effects are described in more detail in U.S. patent application Ser. No. 16/885,596, filed May 28, 2020, published as US2020/0390358A1. These applications and corresponding U.S. publications are incorporated herein by reference in their entirety.

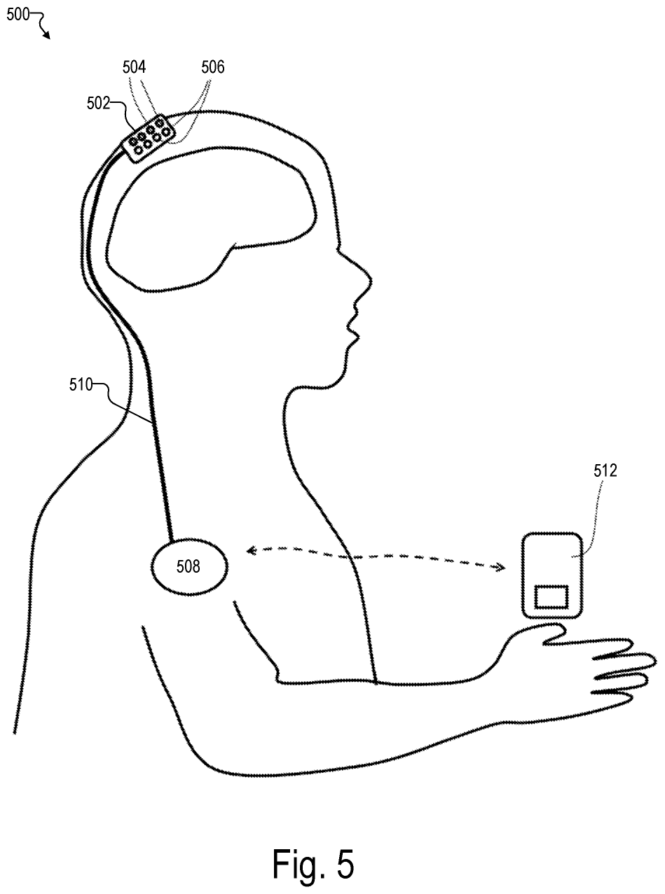

FIG. 5 shows an exemplary non-invasive wearable brain interface system 500 (“brain interface system 500”) that implements optical measurement system 100 (shown in FIG. 1). As shown, brain interface system 500 includes a head-mountable component 502 configured to be attached to a user's head. Head-mountable component 502 may be implemented by a cap shape that is worn on a head of a user. Alternative implementations of head-mountable component 502 include helmets, beanies, headbands, other hat shapes, or other forms conformable to be worn on a user's head, etc. Head-mountable component 502 may be made out of any suitable cloth, soft polymer, plastic, hard shell, and/or any other suitable material as may serve a particular implementation. Examples of headgears used with wearable brain interface systems are described more fully in U.S. Pat. No. 10,340,408, incorporated herein by reference in its entirety.

Head-mountable component 502 includes a plurality of detectors 504, which may implement or be similar to detector 104, and a plurality of light sources 506, which may be implemented by or be similar to light source 110. It will be recognized that in some alternative embodiments, head-mountable component 502 may include a single detector 504 and/or a single light source 506.

Brain interface system 500 may be used for controlling an optical path to the brain and for transforming photodetector measurements into an intensity value that represents an optical property of a target within the brain. Brain interface system 500 allows optical detection of deep anatomical locations beyond skin and bone (e.g., skull) by extracting data from photons originating from light source 506 and emitted to a target location within the user's brain, in contrast to conventional imaging systems and methods (e.g., optical coherence tomography (OCT)), which only image superficial tissue structures or through optically transparent structures.

Brain interface system 500 may further include a processor 508 configured to communicate with (e.g., control and/or receive signals from) detectors 504 and light sources 506 by way of a communication link 510. Communication link 510 may include any suitable wired and/or wireless communication link. Processor 508 may include any suitable housing and may be located on the user's scalp, neck, shoulders, chest, or arm, as may be desirable. In some variations, processor 508 may be integrated in the same assembly housing as detectors 504 and light sources 506.

As shown, brain interface system 500 may optionally include a remote processor 512 in communication with processor 508. For example, remote processor 512 may store measured data from detectors 504 and/or processor 508 from previous detection sessions and/or from multiple brain interface systems (not shown). Power for detectors 504, light sources 506, and/or processor 508 may be provided via a wearable battery (not shown). In some examples, processor 508 and the battery may be enclosed in a single housing, and wires carrying power signals from processor 508 and the battery may extend to detectors 504 and light sources 506. Alternatively, power may be provided wirelessly (e.g., by induction).

In some alternative embodiments, head mountable component 502 does not include individual light sources. Instead, a light source configured to generate the light that is detected by detector 504 may be included elsewhere in brain interface system 500. For example, a light source may be included in processor 508 and coupled to head mountable component 502 through optical connections.

Optical measurement system 100 may alternatively be included in a non-wearable device (e.g., a medical device and/or consumer device that is placed near the head or other body part of a user to perform one or more diagnostic, imaging, and/or consumer-related operations). Optical measurement system 100 may alternatively be included in a sub-assembly enclosure of a wearable invasive device (e.g., an implantable medical device for brain recording and imaging).

Optical measurement system 100 may be modular in that one or more components of optical measurement system 100 may be removed, changed out, or otherwise modified as may serve a particular implementation. Additionally or alternatively, optical measurement system 100 may be modular such that one or more components of optical measurement system 100 may be housed in a separate housing (e.g., module) and/or may be movable relative to other components. Exemplary modular multimodal measurement systems are described in more detail in U.S. Provisional patent application Ser. No. 17/176,460, filed Feb. 16, 2021, U.S. Provisional patent application Ser. No. 17/176,470, filed Feb. 16, 2021, U.S. Provisional patent application Ser. No. 17/176,487, filed Feb. 16, 2021, U.S. Provisional Patent Application No. 63/038,481, filed Feb. 16, 2021, and U.S. Provisional patent application Ser. No. 17/176,560, filed Feb. 16, 2021, which applications are incorporated herein by reference in their respective entireties.

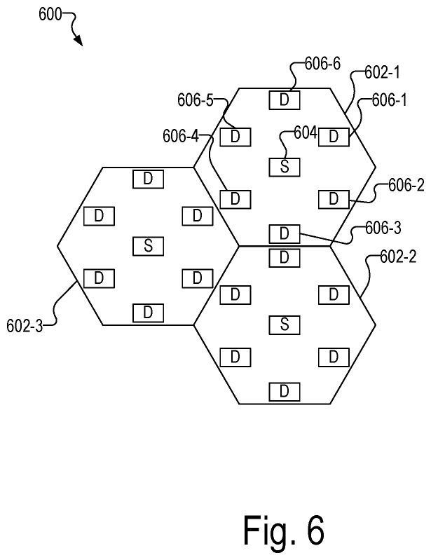

To illustrate, FIG. 6 shows an exemplary wearable module assembly 600 (“assembly 600”) that implements one or more of the optical measurement features described herein. Assembly 600 may be worn on the head or any other suitable body part of the user. As shown, assembly 600 may include a plurality of modules 602 (e.g., modules 602-1 through 602-3). While three modules 602 are shown to be included in assembly 600 in FIG. 6, in alternative configurations, any number of modules 602 (e.g., a single module up to sixteen or more modules) may be included in assembly 600. Moreover, while modules 602 are shown to be adjacent to and touching one another, modules 602 may alternatively be spaced apart from one another (e.g., in implementations where modules 602 are configured to be inserted into individual slots or cutouts of the headgear). Moreover, while modules 602 are shown to have a hexagonal shape, modules 602 may alternatively have any other suitable geometry (e.g., in the shape of a pentagon, octagon, square, rectangular, circular, triangular, free-form, etc.). Assembly 600 may conform to three-dimensional surface geometries, such as a user's head. Exemplary wearable module assemblies comprising a plurality of wearable modules are described in more detail in U.S. Provisional Patent Application No. 62/992,550, filed Mar. 20, 2020, which application is incorporated herein by reference in its entirety.

Each module 602 includes a source 604 and a plurality of detectors 606 (e.g., detectors 606-1 through 606-6). Source 604 may be implemented by one or more light sources similar to light source 110. Each detector 606 may implement or be similar to detector 104 and may include a plurality of photodetectors (e.g., SPADs) as well as other circuitry (e.g., TDCs). As shown, detectors 606 are arranged around and substantially equidistant from source 604. In other words, the spacing between a light source (i.e., a distal end portion of a light source optical conduit) and the detectors (i.e., distal end portions of optical conduits for each detector) are maintained at the same fixed distance on each module to ensure homogeneous coverage over specific areas and to facilitate processing of the detected signals. The fixed spacing also provides consistent spatial (lateral and depth) resolution across the target area of interest, e.g., brain tissue. Moreover, maintaining a known distance between the light emitter and the detector allows subsequent processing of the detected signals to infer spatial (e.g., depth localization, inverse modeling) information about the detected signals. Detectors 606 may be alternatively disposed as may serve a particular implementation.

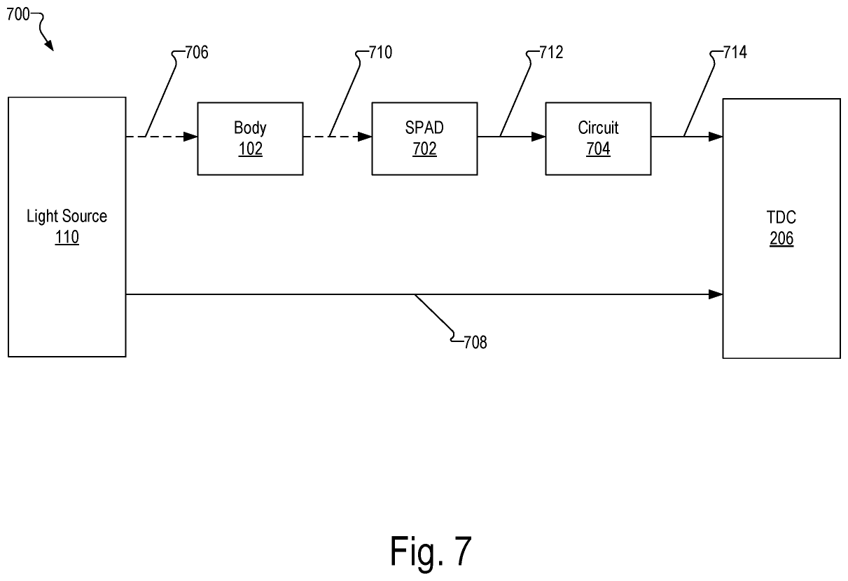

FIG. 7 illustrates an exemplary diagram 700 showing signal paths for components of an optical measurement system (e.g., optical measurement system 100 implementing architecture 200). Diagram 700 includes light source 110 emitting a light pulse to body 102. Diagram 700 further includes a SPAD 702 and a circuit 704, which may be components of a SPAD circuit (e.g., SPAD circuit 202). Diagram 700 also includes TDC 206.

Diagram 700 shows an optical signal 706 output by light source 110, in a form of a light pulse emitted by light source 110 to body 102. Light source 110 concurrently outputs an electrical signal 708 to TDC 206 indicating that light source 110 has emitted the light pulse. Electrical signal 708 may be an implementation of light pulse timing information that TDC 206 uses to start a timer to measure a time that SPAD circuit 202 detects a photon from the light pulse.

Body 102 may scatter (e.g., reflect) photons from the light pulse, shown as an optical signal 710 output by body 102. SPAD 702 detects optical signal 710 (e.g., a photon) and outputs an electrical signal 712 in response. Circuit 704 may be a portion of SPAD circuit 202 that is configured to output an electrical signal 714 in response to the output electrical signal 712 by SPAD 702. TDC 206 receives electrical signal 714 from circuit 704 and records a time as the time SPAD circuit 202 detects the photon from the light pulse. Thus, via the signal path shown in diagram 700, the time that SPAD circuit 202 detects a photon after a light pulse from light source 110 may be measured.

However, each of signals 706-714 shown in diagram 700 may introduce a propagation delay in the measured time, as each signal takes a non-zero amount of time to travel from a source to a destination. Furthermore, a duration of each delay may be variable and depend upon conditions that some of which may themselves be variable, such as temperature, distance between components, etc. Thus, the time ultimately measured by TDC 206 may include an accumulation of nontrivial delays, which may result in errors in histograms and/or other data generated based on the measured time.

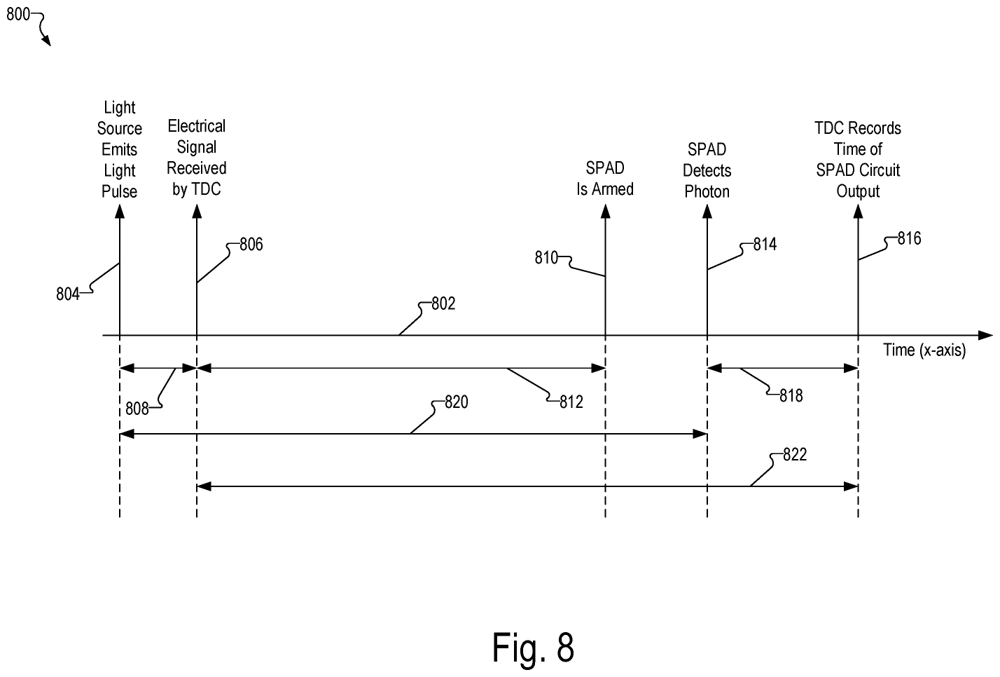

For instance, FIG. 8 shows an exemplary timing diagram 800 that illustrates relative times of events shown in the signal path of diagram 700 and delays introduced in the signal path. Diagram 800 includes an x-axis 802 that represents time. At a first time 804, light source 110 may emit a light pulse. At this first time 804, optical signal 706 and electrical signal 708 (e.g., an electrical pulse) may be output concurrently by light source 110. At a second time 806, electrical signal 708 is received by TDC 206. As electrical signal 708 takes time to travel from light source 110 to TDC 206, a first delay 808 is introduced that is a delay between an actual emission of a light pulse by light source 110 (time 804) and a recorded emission of the light pulse (time 806).

At a third time 810, SPAD 702 is armed, such as by a fast-gating circuit of SPAD circuit 202. SPAD 702 may be configured to be armed after a gate delay 812, for instance, so that SPAD 702 may detect photons scattered by a target of interest in body 102. Thus, time 810 would occur a duration of gate delay 812 after time 806, when the light pulse timing information is received (e.g., by control circuit 204).

At a fourth time 814, SPAD 702 detects a photon, shown in diagram 700 as receiving optical signal 710. At a fifth time 816, TDC 206 records a time that SPAD circuit 202 detected the photon. As shown, a second delay 818 is introduced based on a time electrical signal 712 takes to travel from SPAD 702 to circuit 704 and a time electrical signal 714 takes to travel from circuit 704 to TDC 206. Thus, delay 818 is a delay between an actual detection of a photon by SPAD 702 (time 814) and a recorded detection of the photon (time 816).

Therefore, diagram 800 shows that an actual time of interest is represented by a duration 820 which is a length of time between an actual emission of the light pulse by light source 110 (time 804) to an actual detection of a photon by SPAD 702 (time 814). However, the time that is measured is duration 822, which is a length of time between a recorded emission of the light pulse (time 806) to a recorded detection of the photon (time 816).

While diagram 800 shows timing of the signal path as shown in diagram 700, in some examples, delay 808 may be inverted. Namely, rather than light source 110 outputting electrical signal 708 to indicate that the light pulse has been emitted, in some examples, light source 110 may be provided an electrical signal as an input command to emit the light pulse. In such examples, the electrical signal provided to light source 110 would also concurrently be provided to TDC 206, and the electrical signal would precede the optical signal output by light source 110. Nonetheless, the time measured as the detection of the photon (duration 822) would still be affected (though differently) by such an inverted delay 808 relative to the actual time of interest (duration 820).

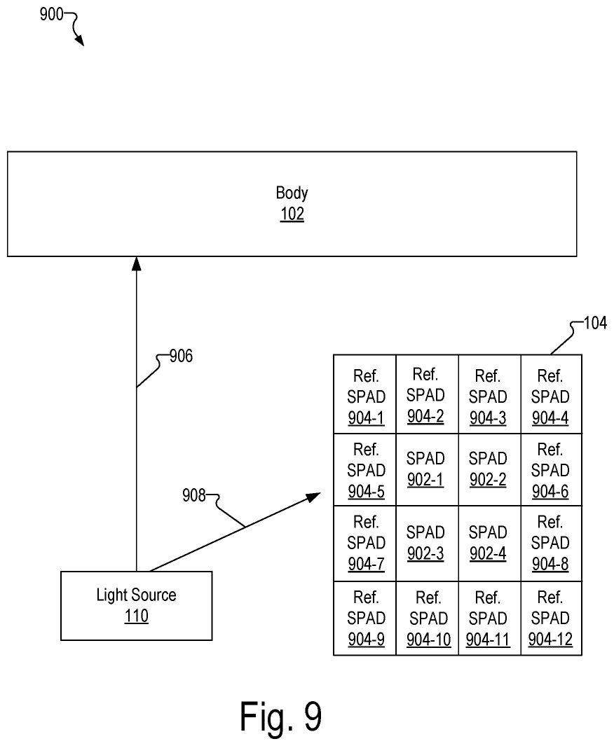

FIG. 9 illustrates an exemplary configuration 900 of components of an optical measurement system (e.g., optical measurement system 100).

Configuration 900 includes light source 110, body 102, and detector 104. As shown, detector 104 includes SPADs 902 and reference SPADs 904 (e.g., SPADs 902-1 through 902-4 and reference SPADs 904-1 through 904-12, which may each be an implementation of photodetector 106). Reference SPADs 904 may be any SPADs that are designated by optical measurement system 100 to be used for determining and/or compensating for propagation delays. Any one or more of the SPADs in detector 104 may be designated as reference SPADs 904 in any suitable layout and/or combination. For example, reference SPADs 904 may be proximate to SPADs 902.

Light source 110 is configured to emit a light pulse that is directed toward both body 102 (shown by an arrow 906) and detector 104 (shown by an arrow 908). Light source 110 may be configured to direct the light pulse at both targets by emitting multiple light pulses concurrently and/or using lenses, mirrors, and/or any other suitable components to split the light pulse into two (or more) directions. Reference SPADs 904 may be configured to be armed and remain armed so that reference SPADs 904 may detect photons from the light pulse directed at detector 104. SPADs 902 may be configured to be armed after a gate delay, so that SPADs 902 may detect photons from the light pulse reflected off of body 102 rather than the light pulse directed at detector 104. Optical measurement system 100 may be configured to use times measured by one or more of reference SPADs 904 to compensate for the delays shown in timing diagram 800 (FIG. 8) with respect to the optical signal outputs and components shown in FIG. 7.

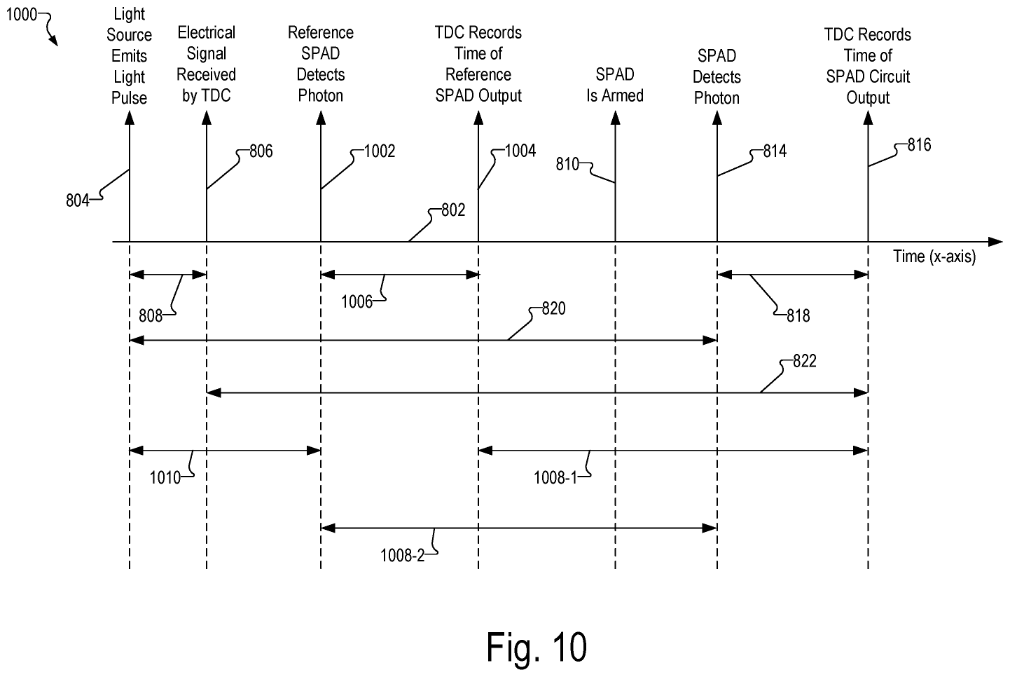

For instance, FIG. 10 shows an exemplary timing diagram 1000 that illustrates relative times of events from configuration 900. Diagram 1000 also includes the events of timing diagram 800 (FIG. 8) with respect to the optical signal outputs and components shown in FIG. 7. Thus, at time 804, light source 110 emits the light pulse. At time 806, electrical signal 708 is received by TDC 206. At time 810, SPAD 702 is armed. SPAD 702 may be implemented by any of SPADs 902 (shown in FIG. 9), detecting photons from the light pulse reflected off of body 102. At time 814, SPAD 702 detects a photon. At time 816, TDC 206 records a time that SPAD circuit 202 detected the photon. Diagram 1000 also includes delay 808, the time between an actual emission of a light pulse by light source 110 (time 804) and a recorded emission of the light pulse (time 806), and delay 818, the time between an actual detection of a photon by SPAD 702 (time 814) and a recorded detection of the photon (time 816). Diagram 1000 also shows duration 820, the actual time of interest, and duration 822, the time measured as the detection of the photon by SPAD 702.

Diagram 1000 also includes a time 1002, which shows when one of reference SPADs 904 detects a photon from the light pulse directly from light source 110. Diagram 1000 also includes a time 1004, which shows when TDC 206 records a time that reference SPAD 904 detected the photon. A delay 1006 shows a time between an actual detection of a photon by reference SPAD 904 (time 1002) and a recorded detection of the photon (time 1004). Delay 1006 may be based on a time an electrical signal takes to travel from reference SPAD 904 to a corresponding reference SPAD circuit and a time the electrical signal takes to travel from the reference SPAD circuit to TDC 206.

As delay 1006 may be based on such a circuit delay of reference SPAD 904, delay 1006 may be considered to be substantially a same duration as delay 818, which is based on similar factors in SPAD 702. Moreover, conditions that may affect the duration of delay 818 would similarly affect the duration of delay 1006, leaving the durations of delay 818 and delay 1006 to be substantially the same. As a result, a difference in time 816 (recorded detection of the photon by SPAD 702) and time 1004 (recorded detection of the photon by reference SPAD 904), which is shown as duration 1008-1, would be equal to a difference in time 814 (actual detection of the photon by SPAD 702) and time 1002 (actual detection of the photon by reference SPAD 904), shown as duration 1008-2. As duration 1008-1 may be calculated, since time 1004 and time 816 are recorded by TDC 206, duration 1008-2 may be determined based on duration 1008-1.

Additionally, a duration 1010 shows a difference between time 1002 (actual detection of the photon by reference SPAD 904) and time 804 (actual emission of the light pulse by light source 110). Duration 1010 may be calculated, as duration 1010 would correspond to a time for light to travel between light source 110 and reference SPAD 904. As the distance between light source 110 and reference SPAD 904 may be predetermined or otherwise known, the known distance and the speed of light, duration 1010 may be determined.

Finally, combining duration 1008 and duration 1010 results in duration 820 (the actual time of interest). As duration 1010 covers time 804 (actual emission of the light pulse) to time 1002 (reference SPAD 904 actually detects the photon) and duration 1008 is equal to time 1002 (reference SPAD 904 actually detecting the photon) to time 814 (SPAD 702 actually detecting the photon), combining the two durations 1010 and 1008 results in the time from light source 110 actually emitting the light pulse to SPAD 702 actually detecting the photon. Thus, by using one or more of reference SPADs 904, optical measurement system 100 may compensate for the delays of the signal paths of diagram 700.

Additionally, reference SPADs 904 may be used to synchronize multiple SPAD arrays, which may be in different locations (e.g., near different portions of body 102). For instance, a first SPAD array may include SPADs 902 and reference SPADs 904 (proximate to SPADs 902 of the first SPAD array), which may be used to compensate for delays of signal paths in the first SPAD array. A second SPAD array may include SPADs 902 and reference SPADs 904 (proximate to SPADs 902 of the second SPAD array), which may be used to compensate for delays of signal paths in the second SPAD array. In addition, the first reference SPADs 904 and the second reference SPADs 904 may be used to synchronize the first SPAD array and the second SPAD array. As reference SPADs 904 in both SPAD arrays are configured to detect photons directly from a light source, reference SPADs 904 in both arrays may both detect the photons at a same time, especially as the light source may be configured to be a same distance from the first SPAD array and the second SPAD array. Thus, any differences in recorded photon detection times between reference SPADs 904 in the first SPAD array and the second SPAD array may be used to determine an offset for SPADs 902 for the SPAD arrays and/or otherwise synchronize the SPAD arrays. In some examples, the light source may be configured to be a different distance from the first SPAD array and the second SPAD array. In such examples, the recorded photon detection times for reference SPADs 904 may be used with the known distances between the light source and each SPAD array to synchronize the SPAD arrays.

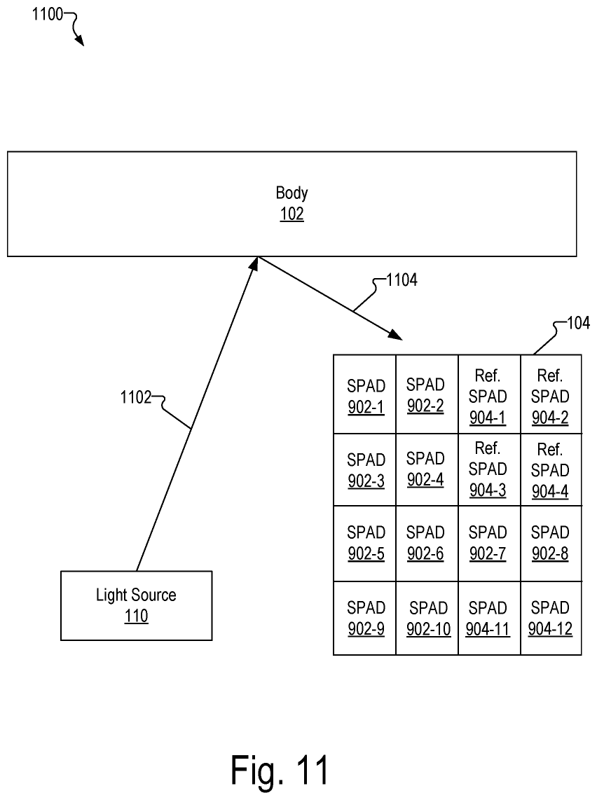

FIG. 11 illustrates another exemplary configuration 1100 of components of an optical measurement system (e.g., optical measurement system 100). Similar to configuration 900, configuration 1100 includes light source 110, body 102, and detector 104. Detector 104 includes SPADs 902 and reference SPADs 904. However, unlike configuration 900, detector 104 includes SPADs 902-1 through 902-12 and reference SPADs 904-1 through 904-4. Rather than light source 110 being configured to emit the light pulse directly at detector 104 and as well as at body 102 (as shown in FIG. 9), in configuration 1100, light source 110 is configured to emit the light pulse directly to body 102 (shown by arrow 1102). Reference SPADs 904 may be configured to be armed and remain armed so that reference SPADs 904 may detect photons from the light pulse that reflect off of a surface external to body 102 (shown by arrow 1104), such as hair or a scalp of body 102. Such photons may arrive at reference SPADs 904 sooner than photons to SPADs 902, as SPADs 902 may be configured to detect photons that reflect off of a target within body 102. For example, SPADs 902 may be configured to be armed after a gate delay so that SPADs 902 detect the photons that reflect off of the target within body 102 rather than off of the surface external to body 102.

Similar to configuration 900 (shown in FIG. 9), the components of optical measurement system 100 shown in FIG. 11 may be configured to use times measured by one or more of reference SPADs 904 to compensate for the delays shown in timing diagram 800 (FIG. 8) with respect to the optical signal outputs and components shown in FIG. 7. As the distance from light source 110 to the surface external to body 102 to reference SPADs 904 may be predetermined, a light path from light source 110 reflecting off the surface external to body 102 to reference SPADs 904 may be used in a manner similar to a light path from light source 110 directly to reference SPADs 904 to compensate for propagation delays as described with reference to configuration 900 and timing diagram 1000.

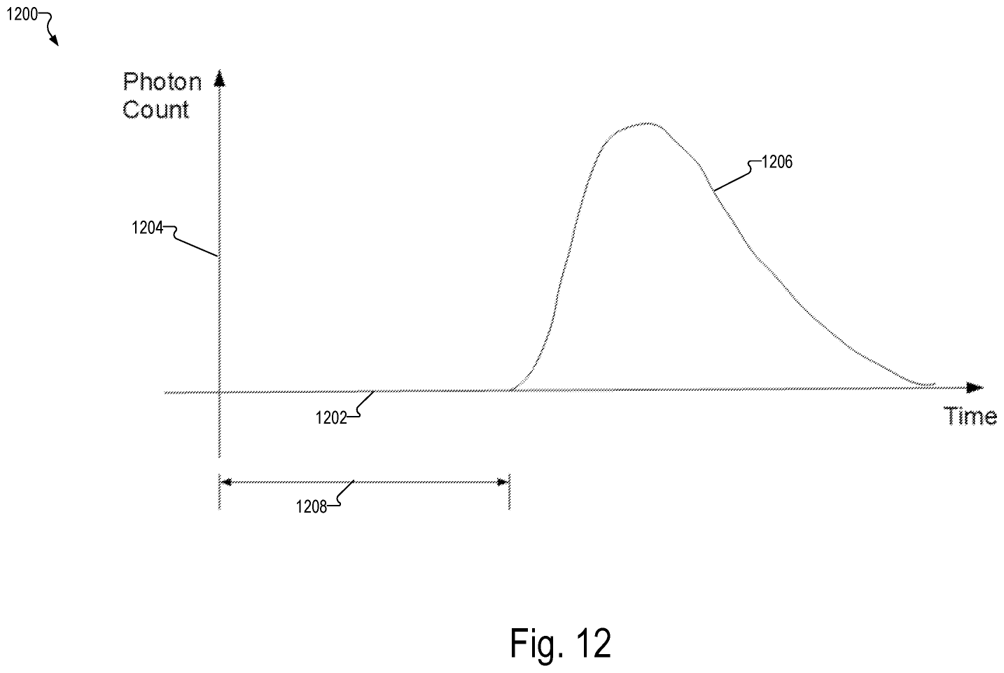

FIG. 12 illustrates an exemplary histogram 1200 (e.g., an implementation of TPSF 402 (see FIG. 4)), based on which optical measurement system 100 may compensate for propagation delays and/or other delays in optical measurement system 100. Histogram 1200 includes an x-axis 1202 that represents time and a y-axis 1204 that represents a photon count. A curve 1206 shows relative photon counts plotted against relative times. A duration 1208 shows a propagation delay based on one or more circuits of optical measurement system 100 as described herein.

Histogram 1200 may be based on photons detected by optical measurement system 100 (e.g., detector 104) that are reflected off of body 102. Curve 1206 may provide a basis for which different targets and/or regions of interest within body 102 may be determined. For instance, a peak of curve 1206 may indicate a specific target on body 102, such as an external surface, from which a majority of photons may be reflected. Specific times after the peak of curve 1206 may indicate specific depths of targets within body 102. Depending on what target within body 102 is of interest, different points on curve 1206 may correspond to windows of time that photodetectors 106 are gated to detect photons from the target of interest.

Such points on curve 1206 (e.g., the peak of curve 1206, a specified time after the peak of curve 1206, a point that is at a specified percentage of photons of the peak of curve 1206, or any other point of interest) may have expected corresponding times that would be expected given parameters of optical measurement system 100 and body 102. However, due to the propagation delays, which may be variable, the actual corresponding times to points on curve 1206 may be offset from the expected corresponding times by the propagation delay. Thus, by generating histogram 1200 based on actual measurements, the propagation delay may be empirically determined. As shown, duration 1208 depicts the propagation delay for optical measurement system 100. Therefore, by appending duration 1208 or an offset based on duration 1208 to the expected corresponding time of a point of interest on curve 1206, an actual time window that corresponds to photons reflecting off of the target within body 102 may be determined and used for arming photodetectors 106.

Histogram 1200 may be generated in any suitable manner. For instance, photodetectors 106 may be armed at a start of a calibration period and left armed in a free running mode until the end of the calibration period. During the calibration period, one or more light pulses may be directed at body 102 and a temporal distribution of photons detected by photodetectors 106 during the calibration period may be plotted relative to a time of an emission of the light pulse.

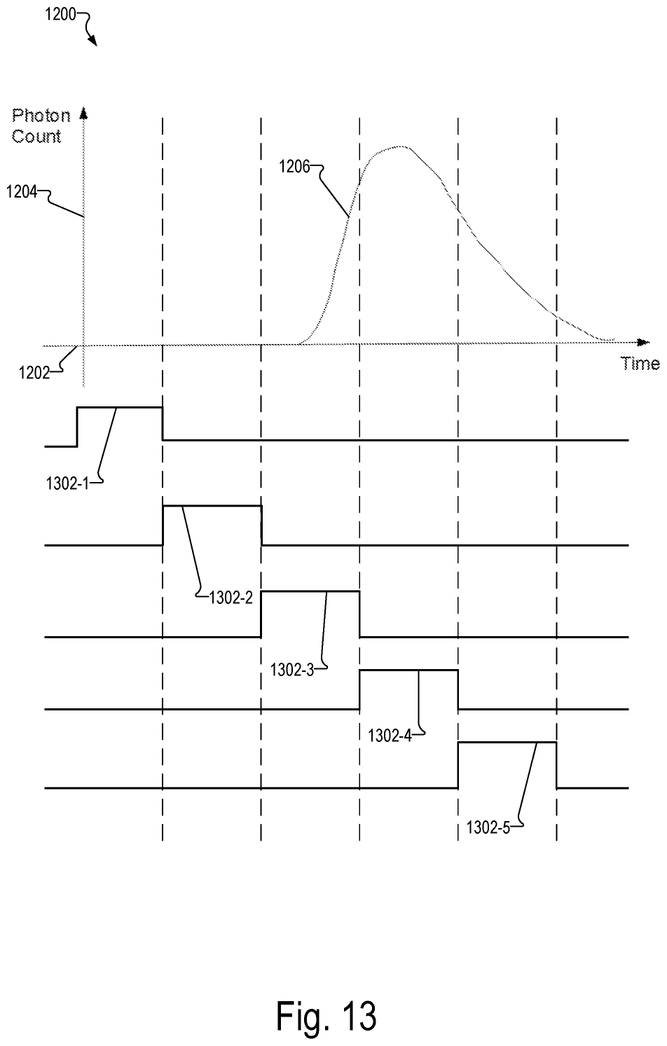

As another example, FIG. 13 illustrates an exemplary method for determining a time window corresponding to the peak of histogram 1200. FIG. 13 shows histogram 1200 divided into a plurality of gate durations 1302 (e.g., gate durations 1302-1 through 1302-5). While histogram 1200 illustrates the temporal distribution of photons in response to a light pulse, histogram 1200 may be generated based on an accumulation of responses to a plurality of light pulses. Thus, the time represented by x-axis 1202 may be based on a cycle duration that separates one light pulse from another in the plurality of light pulses. The cycle duration may be divided into gate durations 1302. Gate durations 1302 may be determined by a length of time that photodetectors 106 are to be armed and disarmed when detecting photons reflected off of the target of interest, e.g., user's brain, within body 102, e.g., a user's head. Alternatively, gate durations 1302 may be longer or shorter, which may provide coarser or finer resolution and/or faster or slower calculation times.

Optical measurement system 100 may sweep through the plurality of gate durations 1302 with photodetectors 106 to determine a gate duration that corresponds to the peak of histogram 1200. For example, for a first light pulse (or a first plurality of light pulses), photodetectors 106 may be armed at the starting point of a first gate duration 1302-1 and disarmed at the ending point of gate duration 1302-1. Optical measurement system 100 may record a number of photodetectors that detect a photon during gate duration 1302-1. In this example, that number may be 0. Then, for a second light pulse(s), photodetectors may be armed at the starting point of a second gate duration 1302-2 and disarmed at the ending point of gate duration 1302-2. Optical measurement system 100 may record a number of photodetectors that detect a photon during gate duration 1302-2. This process may be repeated for each of gate durations 1302-1 through 1302-5. While FIG. 13 shows 5 gate durations, any suitable number of gate durations may be used as appropriate for the lengths of the cycle duration and the gate duration.

In this example, photon counts for gate durations 1302 may be zero (0) for gate duration 1302-1, zero (0) for gate duration 1302-2, fifty (50) for gate duration 1302-3, four-hundred (400) for gate duration 1302-4, and one-hundred (100) for gate duration 1302-5. Thus, by sweeping through gate durations 1302, the time window corresponding to the peak of curve 1206 may be determined as the gate duration with a highest photon count, in this case, gate duration 1302-4. Further, the propagation delay may be determined to be at least a length of gate durations 1302-1 and 1302-2, as the photon counts for both are zero (0). Therefore, based on the peak time window and/or the propagation delay windows, photodetectors 106 may be controlled to arm and disarm for a time window corresponding to the target of interest within body 102.

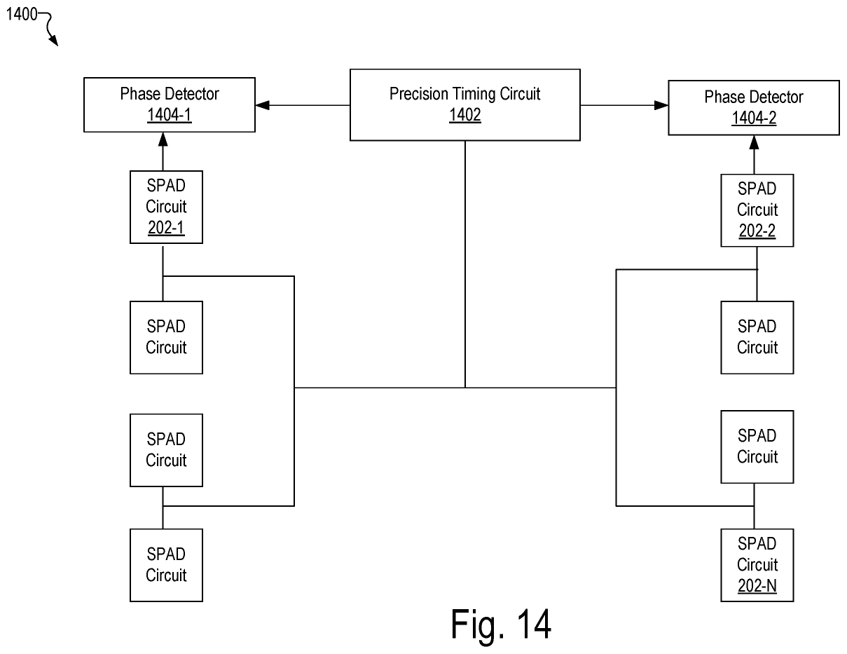

FIG. 14 illustrates an exemplary configuration 1400 of components of an optical measurement system (e.g., optical measurement system 100). Configuration 1400 may be configured to determine a propagation delay in optical measurement system 100 such as a timing signal delay, based on which optical measurement system 100 may compensate for such a delay. Configuration 1400 includes a precision timing circuit 1402 configured to provide signals to SPAD circuits 202 (e.g., SPAD circuit 202-1, 202-2, . . . , through 202-N). Configuration 1400 further includes phase detectors 1404 (e.g., phase detector 1404-1 and phase detector 1404-2).

Precision timing circuit 1402 may be implemented in any suitable manner that allows precision timing circuit 1402 to send signals to SPAD circuits 202 at precise times. Precision timing circuit 1402 may be configured to send gating signals (e.g., signals to arm and disarm SPAD circuits 202), firing signals (e.g., a command for SPAD circuits 202 to fire), reference signals, etc. For instance, precision timing circuit 1402 may be implemented by a portion of controller 112 and/or control circuit 204 or any other suitable component.

SPAD circuits 202 may be configured to be a substantially equal distance from precision timing circuit 1402, which may result in a layout of SPAD circuits 202 with a nontrivial distance between each of SPAD circuits 202 and precision timing circuit 1402 (e.g., an H-tree). Thus, a propagation delay may include a timing signal delay that is a length of time a signal takes to travel from precision timing circuit 1402 to SPAD circuits 202. Further, while precision timing circuit 1402 may be configured to send signals to SPAD circuits 202 concurrently, SPAD circuits 202 may receive the signals at different times due to process variations, temperature variations, or any other such variations in conditions that may affect a time that the signal takes from precision timing circuit 1402 to each of SPAD circuits 202. Therefore, the propagation delay may include a timing signal delay that may vary from SPAD circuit to SPAD circuit.

Configuration 1400 may allow optical measurement system 100 to determine the timing signal delay. For example, precision timing circuit 1402 may be configured to send a calibration signal to SPAD circuit 202-1 and concurrently send a reference signal to phase detector 1404-1. Upon receiving the calibration signal, SPAD circuit 202-1 may be configured to send a response signal to phase detector 1404-1. Phase detector 1404-1 may receive the reference signal from precision timing circuit 1402 and the response signal from SPAD circuit 202-1. Phase detector 1404-1 may compare phases of the reference signal and the response signal to determine a time difference between receiving the reference signal and receiving the response signal. Based on the time difference and a predetermined distance between phase detector 1404-1 and precision timing circuit 1402, phase detector 1404-1 may determine the timing signal delay to SPAD circuit 202-1. Compensating for the timing signal delay may allow optical measurement system 100 to more precisely arm and disarm SPAD circuits 202 at a specific time window relative to a light pulse.

Optical measurement system 100 may further determine different timing signal delays to different ones of SPAD circuits 202 and/or different regions of SPAD circuits 202. For instance, configuration 1400 shows a second phase detector 1404-2 so that precision timing circuit 1402 may send the reference signal also to phase detector 1404-2. Phase detector 1404-2 may also receive a response signal from SPAD circuit 202-2 to determine a timing signal delay to SPAD circuit 202-2. As described, the timing signal delay to SPAD circuit 202-2 may vary from the timing signal delay to SPAD circuit 202-1. Alternatively, the timing signal delays may be substantially the same. While configuration 1400 shows two phase detectors, any suitable number of phase detectors may be distributed throughout to determine timing signal delays from various different SPAD circuits 202 and/or subsets of SPAD circuits 202.

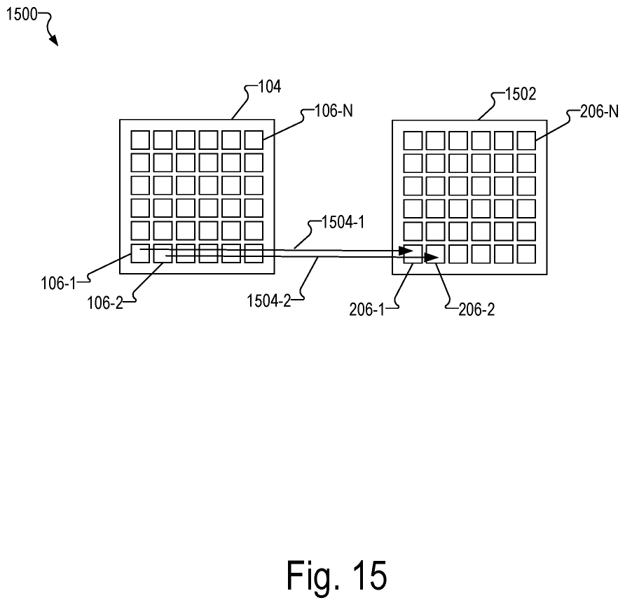

FIG. 15 shows an exemplary configuration 1500 of other components of an optical measurement system (e.g., optical measurement system 100). Configuration 1500 may be configured to determine a propagation delay in optical measurement system 100 such as an offset between photodetector and TDC pairs, based on which optical measurement system 100 may compensate for such a delay. Configuration 1500 includes detector 104, which includes photodetectors 106 (e.g., photodetector 106-1, 106-2, . . . , through photodetector 106-N). Configuration 1500 further includes a TDC array 1502, which includes TDCs 206 (e.g., TDC 206-1, 206-2, . . . , through TDC 206-N).

Each of photodetectors 106 may be configured to send an output signal to a respective one of TDCs 206. For example, photodetector 106-1 may output a signal to TDC 206-1 via a path 1504-1, photodetector 106-2 may output a signal to TDC 206-2 via a path 1504-2, etc. Photodetectors 106 may be configured to output the signal when photodetector 106 detects a photon. Based on receiving the signal, TDC 206 may record a time of the detection of the photon by the respective photodetector 106. However, due to process variations and any other such variations in conditions that may affect a time that the signal takes from photodetector 106 to TDC 206, the times and/or distances for the signal to travel may vary among paths 1504. Thus, even though photodetector 106-1 and photodetector 106-2 may detect respective photons at a same time, TDC 206-1 may record a slightly different time from TDC 206-2, which may result in errors in photon counts.

Optical measurement system 100 may be configured to send a firing signal to photodetectors 106 so that photodetectors 106 output the signal to TDC 206 upon receiving the firing signal. Optical measurement system 100 may send the firing signal simultaneously (or substantially simultaneously) to each of photodetectors 106. Based on the simultaneous sending of the firing signal, times recorded by TDCs 206 as firing times for photodetectors 106 should be a same firing time. Optical measurement system 100 may receive the firing times from each of TDCs 206. For any of TDCs 206 that recorded a time different from a remaining subset of TDCs 206, optical measurement system 100 may determine offsets the corresponding photodetector 106 and TDC 206 pair. In this manner, optical measurement system 100 may compensate for the propagation delay (or offsets in the propagation delay) between photodetectors 106 and TDCs 206.

While various propagation delays and compensations for the various propagation delays have been described herein, in some examples, optical measurement system 100 may combine any or all of the compensations for different propagation delays. For example, optical measurement system 100 may be configured to determine the timing signal delay between precision timing circuit 1402 and SPADs circuits 202. Compensating for differences in the timing signal delay between different SPAD circuits 202 may allow optical measurement system 100 to subsequently determine offsets between photodetector 106 (e.g., SPAD circuit 202) and TDC 206 pairs more accurately. As another example, determining the timing signal delays and/or the offsets between photodetectors 106 and TDCs 206 may allow optical measurement system 100 to generate histograms (e.g., histogram 1200) that more accurately represent photon detection times and thus allow optical measurement system 100 to more accurately determine other system propagation delays.

As mentioned, optical measurement system 100 may be at least partially wearable by a user. For example, optical measurement system 100 may be implemented by a wearable device configured to be worn by a user (e.g., a head-mountable component configured to be worn on a head of the user). The wearable device may include one or more photodetectors and/or any of the other components described herein. In some examples, one or more components (e.g., processor 108, controller 112, etc.) may not be included in the wearable device and/or included in a separate wearable device than the wearable device in which the one or more photodetectors are included. In these examples, one or more communication interfaces (e.g., cables, wireless interfaces, etc.) may be used to facilitate communication between the various components.

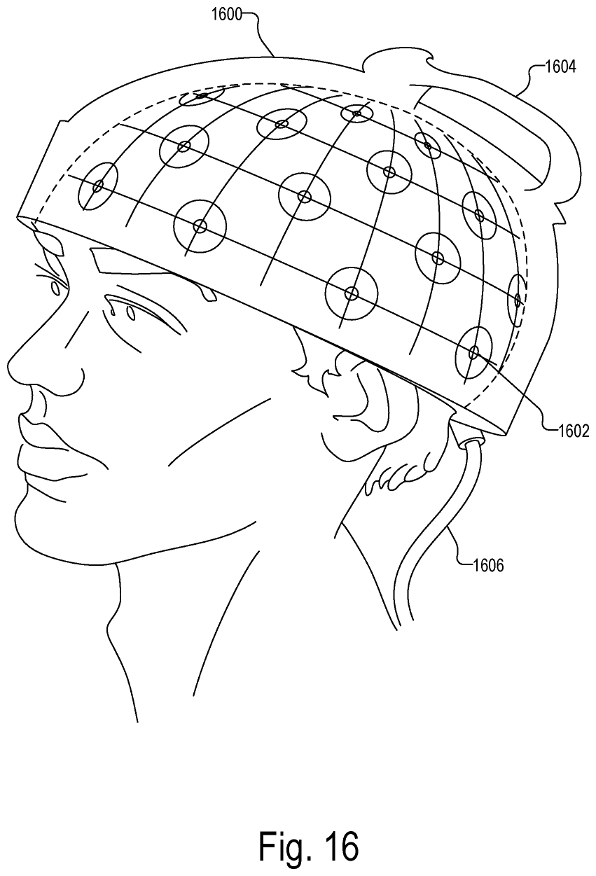

FIGS. 16-21 illustrate embodiments of a wearable device 1600 that includes elements of the optical measurement systems described herein. In particular, the wearable devices 1600 include a plurality of modules 1602, similar to the modules shown in FIG. 6 as described herein. For example, each module 1602 includes a source 604 and a plurality of detectors 606 (e.g., detectors 606-1 through 606-6). Source 604 may be implemented by one or more light sources similar to light source 110. Each detector 606 may implement or be similar to detector 104 and may include a plurality of photodetectors. The wearable devices 1600 may each also include a controller (e.g., controller 112) and a processor (e.g., processor 108) and/or be communicatively connected to a controller and processor. In general, wearable device 1600 may be implemented by any suitable headgear and/or clothing article configured to be worn by a user. The headgear and/or clothing article may include batteries, cables, and/or other peripherals for the components of the optical measurement systems described herein.

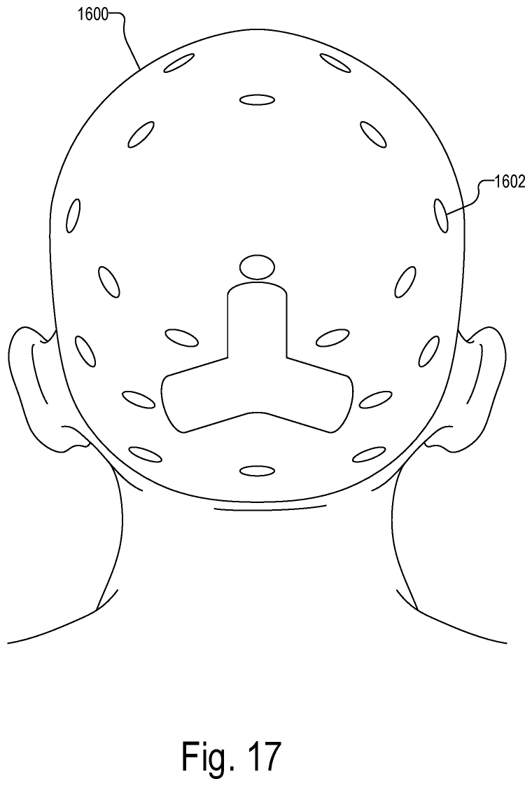

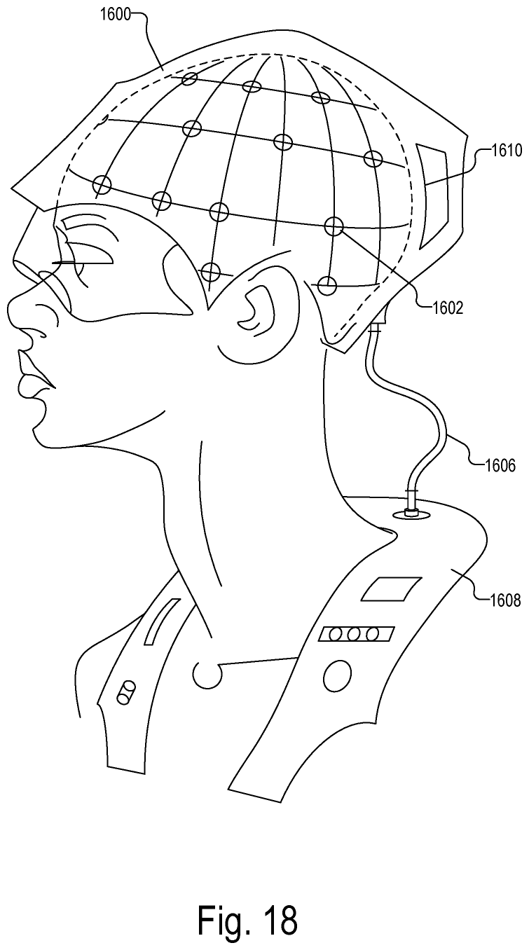

FIG. 16 illustrates an embodiment of a wearable device 1600 in the form of a helmet with a handle 1604. A cable 1606 extends from the wearable device 1600 for attachment to a battery or hub (with components such as a processor or the like). FIG. 17 illustrates another embodiment of a wearable device 1600 in the form of a helmet showing a back view. FIG. 18 illustrates a third embodiment of a wearable device 1600 in the form of a helmet with the cable 1606 leading to a wearable garment 1608 (such as a vest or partial vest) that can include a battery or a hub. Alternatively or additionally, the wearable device 1600 can include a crest 1610 or other protrusion for placement of the hub or battery.

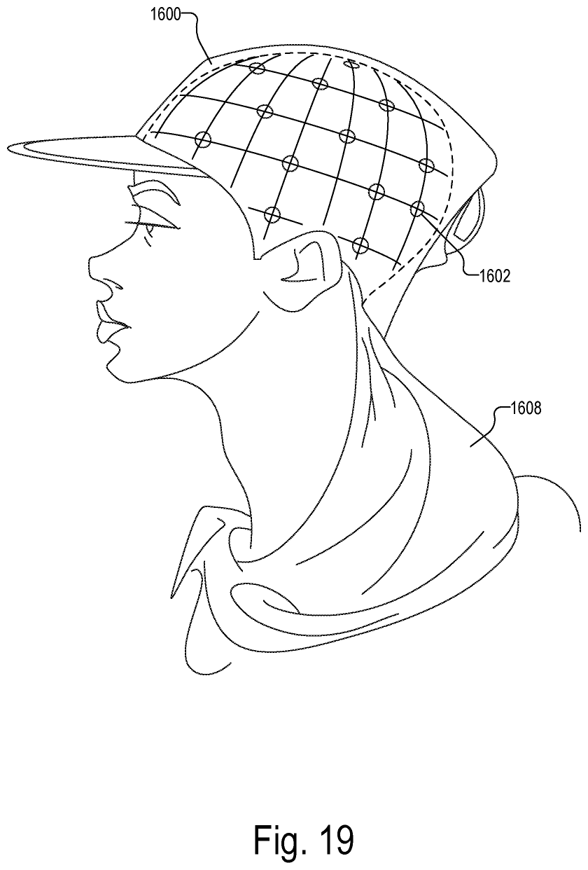

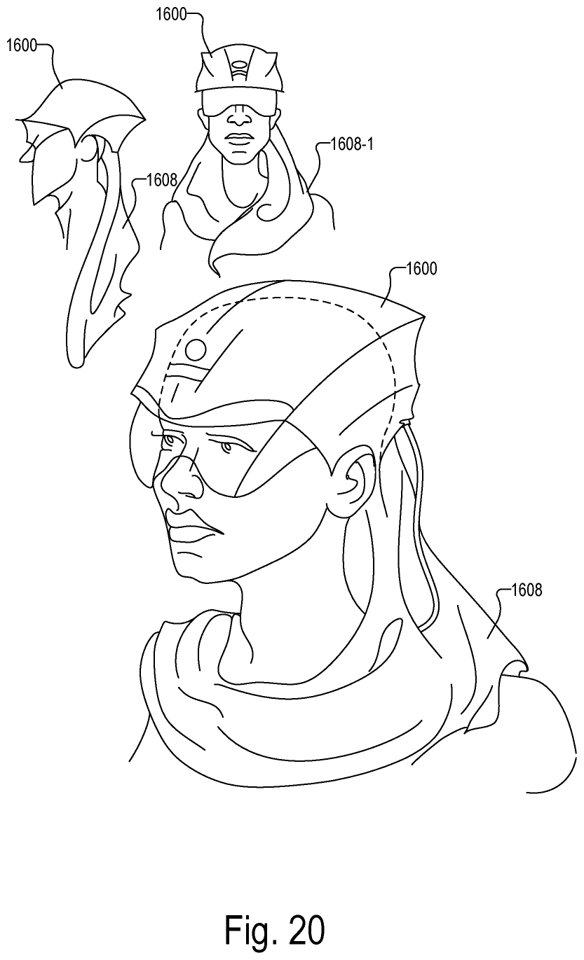

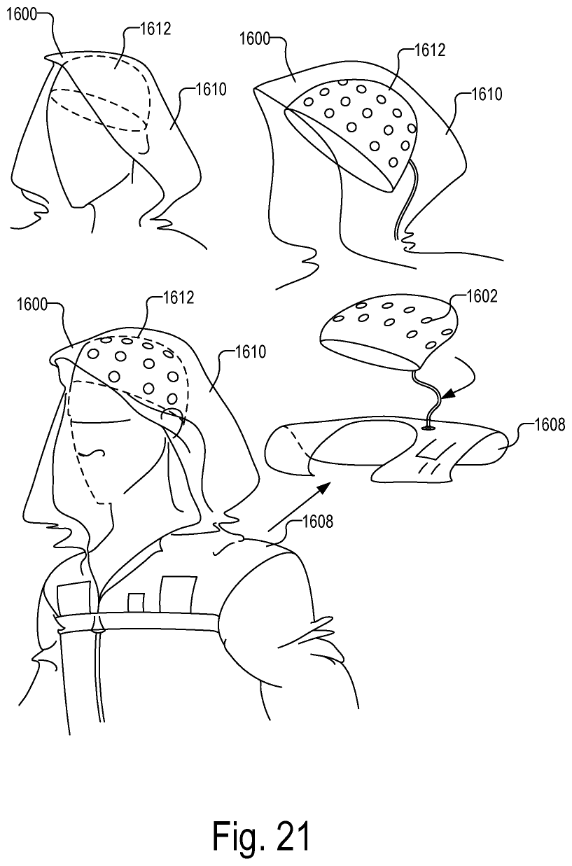

FIG. 19 illustrates another embodiment of a wearable device 1600 in the form of a cap with a wearable garment 1608 in the form of a scarf that may contain or conceal a cable, battery, and/or hub. FIG. 20 illustrates additional embodiments of a wearable device 1600 in the form of a helmet with a one-piece scarf 1608 or two-piece scarf 1608-1. FIG. 21 illustrates an embodiment of a wearable device 1600 that includes a hood 1610 and a beanie 1612 which contains the modules 1602, as well as a wearable garment 1608 that may contain a battery or hub.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

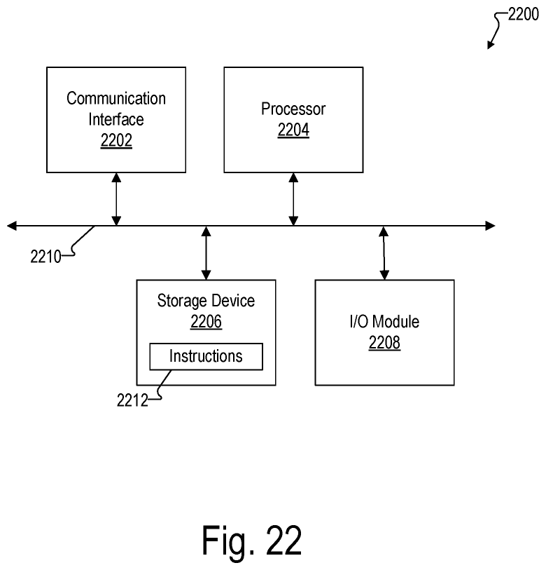

FIG. 22 illustrates an exemplary computing device 2200 that may be specifically configured to perform one or more of the processes described herein. Any of the systems, units, computing devices, and/or other components described herein may be implemented by computing device 2200.

As shown in FIG. 22, computing device 2200 may include a communication interface 2202, a processor 2204, a storage device 2206, and an input/output (“I/O”) module 2208 communicatively connected one to another via a communication infrastructure 2210. While an exemplary computing device 2200 is shown in FIG. 22, the components illustrated in FIG. 22 are not intended to be limiting. Additional or alternative components may be used in other embodiments. Components of computing device 2200 shown in FIG. 22 will now be described in additional detail.

Communication interface 2202 may be configured to communicate with one or more computing devices. Examples of communication interface 2202 include, without limitation, a wired network interface (such as a network interface card), a wireless network interface (such as a wireless network interface card), a modem, an audio/video connection, and any other suitable interface.

Processor 2204 generally represents any type or form of processing unit capable of processing data and/or interpreting, executing, and/or directing execution of one or more of the instructions, processes, and/or operations described herein. Processor 2204 may perform operations by executing computer-executable instructions 2212 (e.g., an application, software, code, and/or other executable data instance) stored in storage device 2206.

Storage device 2206 may include one or more data storage media, devices, or configurations and may employ any type, form, and combination of data storage media and/or device. For example, storage device 2206 may include, but is not limited to, any combination of the non-volatile media and/or volatile media described herein. Electronic data, including data described herein, may be temporarily and/or permanently stored in storage device 2206. For example, data representative of computer-executable instructions 2212 configured to direct processor 2204 to perform any of the operations described herein may be stored within storage device 2206. In some examples, data may be arranged in one or more databases residing within storage device 2206.

I/O module 2208 may include one or more I/O modules configured to receive user input and provide user output. I/O module 2208 may include any hardware, firmware, software, or combination thereof supportive of input and output capabilities. For example, I/O module 2208 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 2208 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 2208 is configured to provide graphical data to a display for presentation to a user. The graphical data may be representative of one or more graphical user interfaces and/or any other graphical content as may serve a particular implementation.

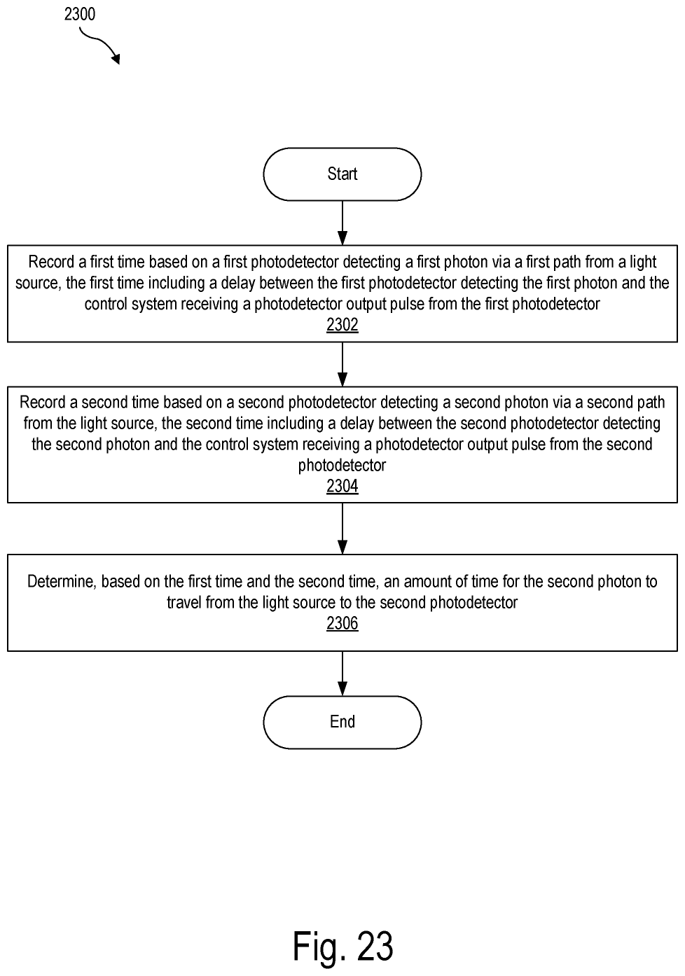

FIG. 23 illustrates an exemplary method 2300 that may be performed by a control system of optical measurement system 100 (e.g., processor 108 and/or controller 112 and/or control circuit 204, etc.) and/or any implementation thereof. While FIG. 23 illustrates exemplary operations according to one embodiment, other embodiments may omit, add to, reorder, and/or modify any of the operations shown in FIG. 23.

In operation 2302, a control system records a first time based on a first photodetector detecting a first photon via a first path from a light source, the first time including a delay between the first photodetector detecting the first photon and the control system receiving a photodetector output pulse from the first photodetector. Operation 2302 may be performed in any of the ways described herein.

In operation 2304, the control system records a second time based on a second photodetector detecting a second photon via a second path from the light source, the second time including a delay between the second photodetector detecting the second photon and the control system receiving a photodetector output pulse from the second photodetector. Operation 2304 may be performed in any of the ways described herein.

In operation 2306, the control system determines, based on the first time and the second time, an amount of time for the second photon to travel from the light source to the second photodetector. Operation 2306 may be performed in any of the ways described herein.

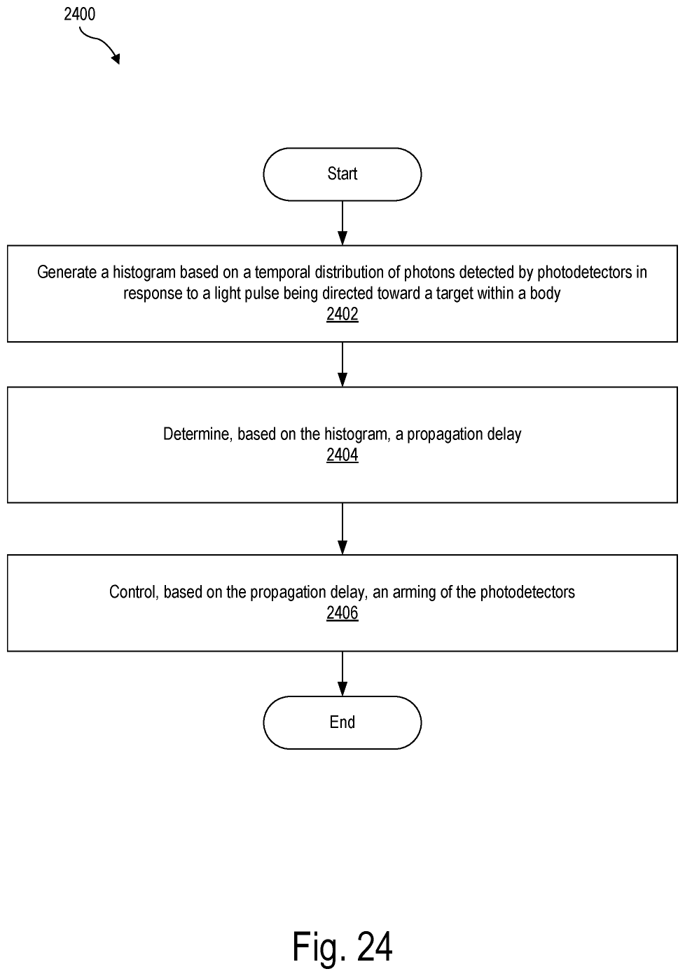

FIG. 24 illustrates an exemplary method 2400 that may be performed by a control system of optical measurement system 100 (e.g., processor 108 and/or controller 112 and/or control circuit 204, etc.) and/or any implementation thereof. While FIG. 24 illustrates exemplary operations according to one embodiment, other embodiments may omit, add to, reorder, and/or modify any of the operations shown in FIG. 24.

In operation 2402, a control system generates a histogram based on a temporal distribution of photons detected by photodetectors in response to a light pulse being directed toward a target within a body. Operation 2402 may be performed in any of the ways described herein.

In operation 2404, the control system determines, based on the histogram, a propagation delay. Operation 2404 may be performed in any of the ways described herein.

In operation 2406, the control system controls, based on the propagation delay, an arming of the photodetectors. Operation 2406 may be performed in any of the ways described herein.

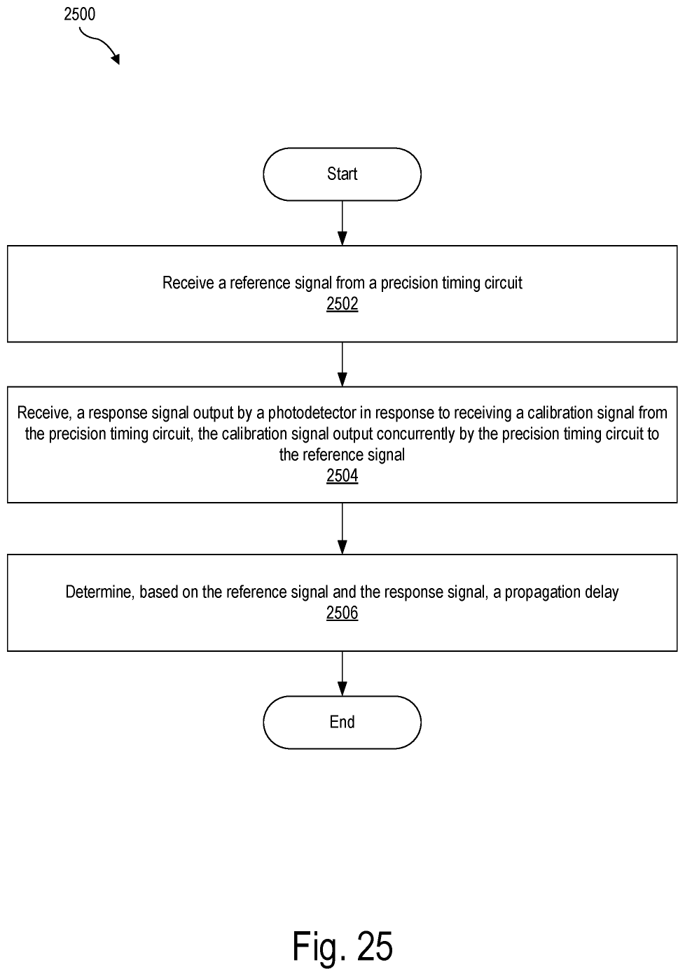

FIG. 25 illustrates an exemplary method 2500 that may be performed by phase detector 1404 and/or any implementation thereof. While FIG. 25 illustrates exemplary operations according to one embodiment, other embodiments may omit, add to, reorder, and/or modify any of the operations shown in FIG. 25.

In operation 2502, a phase detector receives a reference signal from a precision timing circuit. Operation 2502 may be performed in any of the ways described herein.

In operation 2504, the phase detector receives a response signal output by a photodetector in response to receiving a calibration signal from the precision timing circuit, the calibration signal output concurrently by the precision timing circuit to the reference signal. Operation 2504 may be performed in any of the ways described herein.

In operation 2506, the phase detector determines, based on the reference signal and the response signal, a propagation delay. Operation 2506 may be performed in any of the ways described herein.

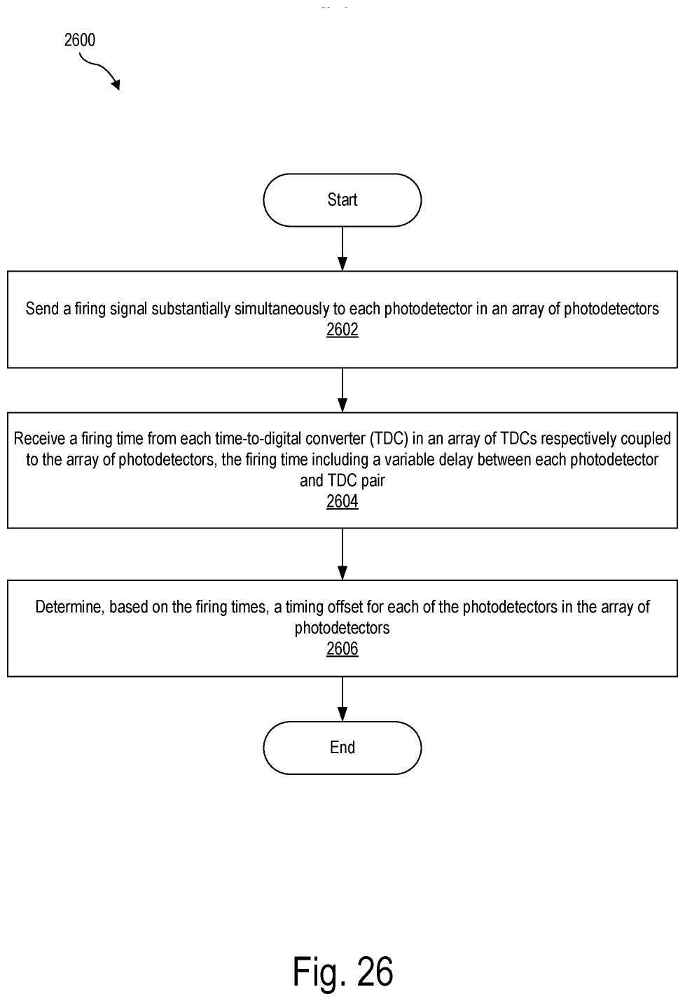

FIG. 26 illustrates an exemplary method 2600 that may be performed by a control system of optical measurement system 100 (e.g., processor 108 and/or controller 112 and/or control circuit 204, etc.) and/or any implementation thereof. While FIG. 26 illustrates exemplary operations according to one embodiment, other embodiments may omit, add to, reorder, and/or modify any of the operations shown in FIG. 26.

In operation 2602, a control system sends a firing signal substantially simultaneously to each photodetector in an array of photodetectors. Operation 2602 may be performed in any of the ways described herein.

In operation 2604, the control system receives a firing time from each TDC in an array of TDCs respectively coupled to the array of photodetectors, the firing time including a variable delay between each photodetector and TDC pair. Operation 2604 may be performed in any of the ways described herein.

In operation 2606, the control system determines, based on the firing times, a timing offset for each of the photodetectors in the array of photodetectors. Operation 2606 may be performed in any of the ways described herein.

An exemplary system includes an array of photodetectors including a first photodetector configured to detect a first photon via a first path from a light source and a second photodetector configured to detect a second photon via a second path from the light source. The system further includes a control system configured to record a first time based on the first photodetector detecting the first photon, the first time including a delay between the first photodetector detecting the first photon and the control system receiving a photodetector output pulse from the first photodetector. The control system is further configured to record a second time based on the second photodetector detecting the second photon, the second time including a delay between the second photodetector detecting the second photon and the control system receiving a photodetector output pulse from the second photodetector. The control system is further configured to determine, based on the first time and the second time, an amount of time for the second photon to travel from the light source to the second photodetector.

An exemplary system includes an array of photodetectors and a control system configured to generate a histogram based on a temporal distribution of photons detected by the photodetectors in response to a light pulse being directed toward a target within a body. The control system is further configured to determine, based on the histogram, a propagation delay of the system. The control system is further configured to control, based on the propagation delay, an arming of the photodetectors.

An exemplary system described herein includes an array of photodetectors including a first photodetector, a phase detector coupled to the first photodetector, and a precision timing circuit configured to concurrently send a calibration signal to the first photodetector and a reference signal to the phase detector. The phase detector is configured to receive a response signal output by the first photodetector in response to receiving the calibration signal from the precision timing circuit. The phase detector is further configured to determine, based on the reference signal and the response signal, a propagation delay.

An exemplary system described herein includes an array of photodetectors and an array of time-to-digital converters (TDCs) respectively coupled to the array of photodetectors. The system further includes a control system configured to send a firing signal substantially simultaneously to each photodetector of the array of photodetectors. The control system is further configured to receive a firing time from each TDC of the array of TDCs, the firing time including a variable delay between each photodetector and TDC pair. The control system is further configured to determine, based on the firing times, a timing offset for each of the photodetectors in the array of photodetectors.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
an array of photodetectors comprising:
a first photodetector configured to detect a first photon via a first path from a light source, and
a second photodetector configured to detect a second photon via a second path from the light source; and
a control system configured to:
record a first time based on the first photodetector detecting the first photon, the first time including a delay between the first photodetector detecting the first photon and the control system receiving a photodetector output pulse from the first photodetector;
record a second time based on the second photodetector detecting the second photon, the second time including a delay between the second photodetector detecting the second photon and the control system receiving a photodetector output pulse from the second photodetector; and
determine, based on the first time and the second time, an amount of time for the second photon to travel from the light source to the second photodetector.

2. The system of claim 1, wherein the determining the amount of time for the second photon to travel from the light source to the second photodetector comprises:
determining a difference between the second time and the first time;
determining a third time based on a distance of the first path; and
combining the third time and the difference between the second time and the first time.

3. The system of claim 1, wherein the first path is a direct path from the light source and the second path includes a reflecting off of a target within a body.

4. The system of claim 1, wherein the first path includes a reflecting off of a surface external to a body and the second path includes a reflecting off of a target within the body.

5. The system of claim 1, wherein the first photodetector is located proximate to the second photodetector, and wherein the array of photodetectors further comprises:
a third photodetector configured to detect a third photon via a third path from the light source; and
a fourth photodetector located proximate to the third photodetector and configured to detect a fourth photon via a fourth path from the light source, and
the control system is further configured to:
record a third time based on the third photodetector detecting the third photon, the third time including a delay between the third photodetector detecting the third photon and the control system receiving a photodetector output pulse from the third photodetector;
record a fourth time based on the fourth photodetector detecting the fourth photon, the fourth time including a delay between the fourth photodetector detecting the fourth photon and the control system receiving a photodetector output pulse from the fourth photodetector;
determine, based on the third time and the fourth time, an amount of time for the fourth photon to travel from the light source to the fourth photodetector; and
determine, based on the first time and the third time, an offset between the amount of time for the second photon to travel from the light source to the second photodetector and the amount of time for the fourth photon to travel from the light source to the fourth photodetector.

6. The system of claim 1, wherein each photodetector of the array of photodetectors comprises:
a single photon avalanche diode (SPAD); and
a fast gating circuit configured to arm and disarm the SPAD.

7. The system of claim 1, wherein the array of photodetectors is included in a wearable device configured to be worn by a user.

8. The system of claim 7, wherein the wearable device includes a head-mountable component configured to be worn on a head of the user.

9. A method comprising:
recording, by a control system, a first time based on a first photodetector detecting a first photon via a first path from a light source, the first time including a delay between the first photodetector detecting the first photon and the control system receiving a photodetector output pulse from the first photodetector;
recording, by the control system, a second time based on a second photodetector detecting a second photon via a second path from the light source, the second time including a delay between the second photodetector detecting the second photon and the control system receiving a photodetector output pulse from the second photodetector; and
determining, by the control system, based on the first time and the second time, an amount of time for the second photon to travel from the light source to the second photodetector.

10. The method of claim 9, wherein the determining the amount of time for the second photon to travel from the light source to the second photodetector comprises:
determining a difference between the second time and the first time;
determining a third time based on a distance of the first path; and
combining the third time and the difference between the second time and the first time.

11-12. (canceled)

13. A system comprising:
an array of photodetectors; and
a control system configured to:
generate a histogram based on a temporal distribution of photons detected by the photodetectors in response to a light pulse being directed toward a target within a body;
determine, based on the histogram, a propagation delay of the system; and
control, based on the propagation delay, an arming of the photodetectors.

14. The system of claim 13, wherein:
the control system is further configured to direct the array of photodetectors to arm at a start of a calibration period and disarm at an end of the calibration period, the calibration period including a plurality of light pulses; and
the generating the histogram includes determining times of photons detected by the photodetectors relative to respective light pulses of the plurality of light pulses.

15. The system of claim 13, wherein the control system is further configured to:
direct a plurality of light pulses toward the target within the body, the plurality of light pulses separated one from another by a cycle duration;
determine a plurality gate durations in the cycle duration;
sweep through the plurality of gate durations with the array of photodetectors by directing the array of photodetectors to arm and disarm for each gate duration of the plurality of gate durations, the directing performed for each gate duration relative to a different light pulse of the plurality of light pulses; and
record, for each gate duration, a number of photodetectors of the array of photodetectors that detect a photon to determine the temporal distribution of photons.

16. The system of claim 13, further comprising:
a phase detector coupled to a first photodetector of the array of photodetectors; and
a precision timing circuit configured to concurrently send a calibration signal to the first photodetector and a reference signal to the phase detector, and wherein:
the phase detector is configured to:
receive a response signal output by the first photodetector in response to receiving the calibration signal from the precision timing circuit, and
determine, based on the reference signal and the response signal, a timing signal delay; and
the controlling the arming of the photodetectors is further based on the timing signal delay.

17. The system of claim 16, further comprising an additional phase detector coupled to a second photodetector of the array of photodetectors, and wherein:
the precision timing circuit is further configured to concurrently send the calibration signal to the second photodetector and the reference signal to the additional phase detector;
the additional phase detector is configured to:
receive a response signal output by the second photodetector in response to receiving the calibration signal from the precision timing circuit, and
determine, based on the reference signal and the response signal output by the second photodetector, an additional timing signal delay; and
the controlling the arming of the photodetectors is further based on the additional timing signal delay.

18. The system of claim 17, further comprising an array of time-to-digital converters (TDCs) respectively coupled to the array of photodetectors, and wherein:
the control system is further configured to:
send, based on the timing signal delay and the additional timing signal delay, a firing signal configured to be received substantially simultaneously to each photodetector of the array of photodetectors,
receive a firing time from each TDC of the array of TDCs, the firing time including a variable delay between each photodetector and TDC pair, and
determine, based on the firing times, a timing offset for each of the photodetectors in the array of photodetectors; and
the temporal distribution of photons detected by the photodetectors is based on the timing offset for each of the photodetectors.

19. The system of claim 13, further comprising an array of TDCs respectively coupled to the array of photodetectors, and wherein:
the control system is further configured to:
send, based on the propagation delay and an additional propagation delay, a firing signal configured to be received substantially simultaneously to each photodetector of the array of photodetectors,
receive a firing time from each TDC of the array of TDCs, the firing time including a variable delay between each photodetector and TDC pair, and
determine, based on the firing times, a timing offset for each of the photodetectors in the array of photodetectors; and
the temporal distribution of photons detected by the photodetectors is based on the timing offset for each of the photodetectors.

20. The system of claim 13, wherein each photodetector of the array of photodetectors comprises:
a single photon avalanche diode (SPAD); and
a fast gating circuit configured to arm and disarm the SPAD.

21. The system of claim 13, wherein the array of photodetectors is included in a wearable device configured to be worn by a user.

22. The system of claim 21, wherein the wearable device includes a head-mountable component configured to be worn on a head of the user.

23-44. (canceled)
